(19) United States
(12) Patent Application Publication
(10) Pub. No.: US 2022/0177874 A1
(43) Pub. Date: Jun. 9, 2022

(54) METHODS FOR LIBRARY PREPARATION TO ENRICH INFORMATIVE DNA FRAGMENTS USING ENZYMATIC DIGESTION

(71) Applicants: The Regents of the University of California, Oakland, CA (US); EarlyDiagnostics, Inc., Los Angeles, CA (US)

(72) Inventors: Xiaohui NI, Los Angeles, CA (US); Xianghong Jasmine ZHOU, Los Angeles, CA (US); Weihua ZENG, Los Angeles, CA (US); Mary Louisa STACKPOLE, Los Angeles, CA (US)

(73) Assignees: The Regents of the University of California, Oakland, CA (US); EarlyDiagnostics, Inc., Los Angeles, CA (US)

(21) Appl. No.: 17/598,021

(22) PCT Filed: Apr. 28, 2020
(86) PCT No.: PCT/US2020/030298

§ 371 Date: Sep. 24, 2021

Related U.S. Application Data
(60) Provisional application No. 62/839,719, filed on Apr. 28, 2019.

(51) Int. Cl. C12N 15/10

(52) U.S. Cl. CPC C12N 15/1065; C12N 15/1093

(57) ABSTRACT

The present disclosure provides methods and compositions for preparation of a nucleic acid library. In some embodiments, the nucleic acids comprise cell-free DNA, including cfDNA that is in need of analysis, such as by sequencing. The methods may comprise restriction enzyme digestion, adapter ligation, and subsequent amplification, and may provide improved approaches for reducing the number adapter dimers produced during the process. In an aspect, a method for preparing a library of nucleic acids may comprise: digesting DNA molecules with restriction enzymes to produce DNA fragments; ligating adapters to the DNA fragments by incubating with ligase to produce a mixture of adapter-ligated DNA fragments and adapter dimers; amplifying the adapter-ligated DNA fragments to produce amplified adapter-ligated DNA fragments; and reducing the quantity of the adapter dimers by differentiating between the junction between an adapter and a DNA fragment, and the junction between an adapter and another adapter.

### CROSS-REFERENCE

This application claims the benefit of U.S. Provisional Patent Application No. 62/839,719, filed Apr. 28, 2019, which is entirely incorporated herein by reference.

### TECHNICAL FIELD

Embodiments of the present disclosure include at least the fields of nucleic acid preparation and analysis, sequencing, molecular biology, cell biology, and medicine.

### BACKGROUND

With the rapid development of next generation sequencing (NGS) technologies, analysis of genomic alterations in deoxyribonucleic acid (DNA) has become a routine analysis to provide diagnostic information about disease (e.g., cancer) or other health (e.g., fetal genetic materials in maternal blood) status. Typical sequencing library preparation techniques may comprise one or more operations, such as DNA fragmentation, end-repair of fragments, dA tailing, adapter ligation, and polymerase chain reaction (PCR) enrichment, as well as one or more purification steps.

Certain health conditions such as cancers or infectious diseases can cause release of DNA into the bloodstream or lymphatic system, where tumor DNA or microbiome DNA may become part of circulating cell-free DNA (cfDNA) in bodily fluids such as plasma or urine. Such cfDNA may be subjected to genomic or epigenomic profiling for clinical applications such as cancer screening, microbial detection, or prenatal testing. For example, whole-genome bisulfite sequencing (WGBS) can provide a comprehensive view of the DNA methylome, but it can be expensive to deep sequence the entire genome. Methods to enrich cell-free DNA for informative regions may advantageously allow genomic or epigenomic profiling towards clinical diagnostic applications. The use of restriction enzymes, clustered regularly interspaced short palindromic repeats (CRISPR), transposase, or other techniques can fragment intact DNA in the way that informative fragments can be enriched by size selection. For example, MspI enzyme digestion can enrich CpG-rich regions by producing smaller fragments that can be used for methylation profiling.

The fragmented nature of cell-free DNA, which may exhibit a characteristic peak around 166 base pairs (bp), poses challenges for typical restriction enzyme digestion-based enrichment approaches. Any size selection process to select informative DNA may select all or nearly all the population of cfDNA and hence result in low enrichment.

The present disclosure provides improvements on methods and compositions for nucleic acid library preparation.

### SUMMARY

The present disclosure provides methods of preparing a nucleic acid library using restriction enzymes and adapters, wherein such preparation methods represent an improvement on technologies. In some embodiments, the methods comprise library preparation with having reduced levels of adapter dimers. Once prepared, the nucleic acid library may be utilized for any purpose, including for next-generation sequencing, for example. In some embodiments, the present disclosure relates to methods of preparing a library from informative deoxyribonucleic acid (DNA) fragments whose sequence, modification status, and/or level are indicative of a medical condition or risk thereof or susceptibility thereto. As used herein, “informative fragments” refers to fragments that have been produced by cutting with a restriction enzyme (for example, multiple CpG sites after MspI restriction enzyme digestion). The nucleic acid may be of any kind, but in some embodiments, the nucleic acid comprises DNA, including cell-free DNA (cfDNA). In some embodiments, the library is utilized for methylation profiling of cfDNA.

In an aspect, the present disclosure provides a method for preparing a library (for example, for the purpose of analyzing including by sequencing) of nucleic acids (e.g., from a plurality of deoxyribonucleic acid (DNA) molecules of a subject), comprising: subjecting the plurality of DNA molecules to enzymatic digestion to fragment at least a subset of the DNA molecules to produce DNA fragments with overhangs at one or both ends; ligating adapters with overhangs that complement the overhangs of the DNA fragments to produce a plurality of tagged DNA molecules; enriching the plurality of tagged DNA molecules (that may be referred to herein as adapter ligated DNA molecules or fragments) before or after reducing the number of adapter dimers; and optionally subjecting the plurality of tagged DNA molecules, or derivatives thereof, to nucleic acid sequencing to yield a plurality of sequence reads.

In some embodiments, restriction enzymes such as BspDI, ClaI, AclI, NarI, Xhol, SmlI, HpyF30I, PaeR71, Sfr274I, or a combination thereof are used to digest adapter dimers during and/or after ligating the adapters. In some embodiments, the subjecting and ligating steps are performed in the same reaction using (1) one or more restriction enzymes, such as MspI and/or HpaII and/or TaqαI, and (2) a ligase, such as T7 and/or T4 ligase. In some embodiments, the subjecting, ligating, and reducing steps are performed in the same reaction using (1) one or more restriction enzymes, such as MspI, and/or HpaII, and/or TaqαI, and/or BspD1, and/or ClaI, and/or AclI, and/or NarI, and/or XhoI, and/or SmlI, and/or HpyF30I, and/or PaeR7I, and/or Sfr274I and (2) a ligase, such as T7 and/or T4 ligase. In some embodiments, the enriching of the plurality of tagged DNA comprises an amplification step, such as with polymerase chain reaction (PCR). In some embodiments, the enriching of the plurality of tagged DNA comprises targeted capture. In some embodiments, the plurality of tagged DNA undergoes bisulfite conversion. In some embodiments, the primer for PCR is designed to recognize the junction between the adapter and targeted DNA but not the junction between adapter and adapter.

In an aspect, the present disclosure provides a method for enriching a plurality of DNA fragments from a plurality of cfDNA molecules of a subject, comprising: subjecting the plurality of cfDNA molecules to enzymatic digestion to fragment at least a subset of the cfDNA molecules to generate fragments that comprise one or more regions of interest; ligating adapters with overhangs that are complementary to the overhangs of the plurality of fragmented cfDNA molecules to provide a plurality of tagged DNA molecules; reducing the number of adapter dimers; optionally subjecting the plurality of tagged DNA molecules or derivatives thereof to nucleic acid sequencing to yield a plurality of sequence reads; and processing the plurality of sequence reads to provide one or more clinical applications.

In another aspect, the present disclosure provides a method for preparing a library of nucleic acids, comprising: (a) providing a plurality of DNA molecules; (b) subjecting the molecules to digestion with a first one or more restriction enzymes, wherein the subjecting produces DNA fragments; (c) subjecting (e.g., ligating) the DNA fragments to adapters, wherein the subjecting produces a mixture of adapter-ligated DNA fragments and adapter dimers; and (d) amplifying the adapter-ligated DNA fragments to produce amplified adapter-ligated DNA fragments, wherein optionally the method further comprises performing (b) and (c) in the same operation in some embodiments, wherein the method further comprises reducing adapter dimers produced, wherein the reducing is performed during or after (c) and/or after (d), wherein the reducing comprises differentiating between the junction between an adapter and a DNA fragment, and the junction between an adapter and another adapter.

In another aspect, the present disclosure provides a method for preparing a library of nucleic acids, comprising: (a) providing a plurality of DNA molecules; (b) subjecting the molecules to digestion with a first one or more restriction enzymes, wherein said subjecting produces DNA fragments; (c) subjecting (e.g., ligating) the DNA fragments to adapters, wherein said subjecting produces a mixture of adapter-ligated DNA fragments and adapter dimers; and (d) amplifying the adapter-ligated DNA fragments to produce amplified adapter-ligated DNA fragments, subject to one or more of the following: (1) performing (d) using a primer that binds a junction between the end of the DNA fragment and the adapter, but does not bind a junction between the end of one adapter and the end of another adapter; (2) subjecting the mixture of adapter-ligated DNA fragments and adapter dimers to a second one or more restriction enzymes that digests the junction between the end of one adapter and the end of another adapter, but does not digest the junction between the end of the DNA fragment and the adapter; (3) performing (b) in the same reaction with (c) in the presence of a second one or more restriction enzymes that digest the junction between the end of one adapter and the end of another adapter, but does not digest the junction between the end of the DNA fragment and the adapter; (4) the adapter is an adapter dimer by design, and a third one or more restriction enzymes digest the junction between the end of one adapter and the end of another adapter, but does not digest the junction between the end of the DNA fragment and the adapter; and/or (5) the amplifying also produces amplified adapter dimers that are digested with a fourth one or more restriction enzymes that digest the junction between the end of one adapter and the end of another adapter.

In some embodiments, subjecting the plurality of cfDNA molecules to enzymatic digestion comprises performing digestion with one or more restriction enzymes on the plurality of cell-free DNA molecules. In some embodiments, the method utilizes one or more restriction enzymes selected from the group consisting of AcII, HindIII, MluCI, PciI, AgeI, BspMI, BfuAI, SexAI, MluI, BceAI, HpyCH4IV, HpyCH4III, BaeI, BsaXI, AflIII, SpeI, BsrI, BmrI, BglII, BspDI, PI-SceI, NsiI, AseI, CspCI, MfeI, BssS^{α}I, DraIII, EcoP15I, AlwNI, BtsIMutI, NdeI, CviAII, FatI, NlaIII, FspEI, XcmI, BstXI, PflMI, BccI, NcoI, BseYI, FauI, TspMI, XmaI, LpnPI, AclI, ClaI, SacII, HpaII, MspI, ScrFI, StyD4I, BsaJI, BslI, BtgI, NciI, AvrII, MnlI, BbvCI, SbfI, Bpu10I, Bsu36I, EcoNI, HpyAV, BstNI, PspGI, StyI, BcgI, PvuI, EagI, RsrII, BsiEI, BsiWI, BsmBI, Hpy99I, AbaSI, MspJI, SgrAI, BfaI, BspCNI, XhoI, PaeR7I, EarI, AcuI, PstI, BpmI, DdeI, SfcI, AflII, BpuEI, SmlI, Aval, BsoBI, MboII, BbsI, BsmI, EcoRI, HgaI, AatII, PflFI, Tth111I, AhdI, DrdI, SacI, BseRI, PleI, HinfI, Sau3AI, MboI, DpnII, TfiI, BsrDI, BbvI, Bts^{α}I, BstAPI, SfaNI, SphI, NmeAIII, NgoMIV, BglI, AsiSI, BtgZI, HhaI, HinPlI, BssHII, NotI, Fnu4HI, MwolI, BmtI, NheI, BspQI, BlpI, TseI, ApeKI, Bsp1286I, AlwI, BamHI, BtsCI, FokI, FseI, SfiI, NarI, PluTI, KasI, AscI, EciI, BsmFI, ApaI, PspOMI, Sau96I, KpnI, Acc65I, BsaI, HphI, BstEII, AvaII, BanI, BaeGI, BsaHI, BanII, CviQI, BciVI, SalI, BcoDI, BsmAI, ApaLI, BsgI, AccI, Tsp45I, BsiHKAI, TspRI, ApoI, NspI, BsrF^{α}I, BstYI, HaeII, EcoO109I, PpuMI, I-CeuI, I-SceI, BspHI, BspEI, MmeI, Taq^{α}I, Hpy188I, Hpy188III, XbaI, MI, PI-PspI, BsrGI, MseI, PacI, BstBI, PspXI, BsaWI, EaeI, HpyF30I, Sfr274I, and a combination thereof. In certain embodiments, it is contemplated that 1, 2, 3, 4, 5, 6, 7, 8, 9, 10 or more, or any range derivable therein, of these may be excluded.

In some embodiments, subjecting the plurality of cfDNA molecules to enzymatic digestion comprises cutting cell-free DNA molecules with CRISPR (clustered regularly interspaced short palindromic repeats)-Cas9 system or functional derivatives thereof. In some embodiments, the subjecting of the plurality of cfDNA molecules to enzymatic digestion comprises cutting the cfDNA molecules with one or more transposases or functional derivatives thereof.

In some embodiments, the method further comprises subjecting the plurality of tagged DNA fragments or derivatives thereof to conditions sufficient to permit distinction between methylated nucleic acid bases and unmethylated nucleic acid bases in the tagged DNA fragments. In some embodiments, subjecting the plurality of tagged DNA fragments or derivatives thereof to conditions to distinguish methylated vs. unmethylated bases comprises performing bisulfite conversion on the plurality of tagged DNA fragments. In some embodiments, subjecting the plurality of tagged DNA fragments or derivatives thereof to conditions to distinguish methylated vs. unmethylated bases comprises enzymatic and/or chemical reactions to oxidize the methylated cytosine nucleic acid bases and/or hydroxymethylated cytosine nucleic acid bases, followed by reduction and/or deamination of oxidation reaction products.

In some embodiments, restriction enzymes, such as BspDI, ClaI, AclI, NarI, Xhol, SmlI, HpyF30I, PaeR7I, and/or Sfr274I, are utilized to digest adapter dimers during and/or after ligation of the adapters and/or after PCR amplification steps and/or after both bisulfite conversion and PCR amplification.

In some embodiments, subjecting the cfDNA to enzymatic digestion and ligation of the adapters are performed in the same reaction. Further, some embodiments, the enzymes used are MspI and/or BspDI, and the ligase may be any ligase, including T7 and/or T4 DNA ligase.

In some embodiments, enriching of the plurality of tagged DNA comprises amplification, such as PCR. In some embodiments, the primer for PCR is designed to recognize (e.g., be able to bind) the junction between the adapter and the targeted DNA, but not recognize the junction between two adapter molecules ligated to each other. In some embodiments, the primer for PCR is designed to recognize the junction between an adapter and targeted DNA after bisulfite conversion, but the primer does not recognize the junction between adapter and adapter after bisulfite conversion. In some embodiments, the primer for PCR is designed to recognize the junction between adapter and targeted DNA after enzymatic and/or chemical reactions to oxidize the methylated cytosine nucleic acid bases and/or hydroxymethylated cytosine nucleic acid bases, followed by reduction and/or deamination of oxidation reaction products, but not to recognize the junction between adapter and adapter after the enzymatic and/or chemical reactions.

The produced libraries may comprise one or more regions of interest may be of any kind. Further, in some embodiments, they comprise one or more CpG sites.

The adapters for ligation onto the DNA fragments may themselves be designed as adapter-adapter dimers, for example, for long term stability.

In some embodiments, the present disclosure provides sequencing library preparation methods that simplify DNA fragmentation and adapter ligation and that reduce adapter dimers. An example of an application of methods of the present disclosure is to profile a cfDNA methylome for cancer diagnosis and screening, following a library preparation method of the present disclosure.

In another aspect, the present disclosure provides a method for preparing a library of nucleic acids, comprising: (a) providing a plurality of DNA molecules; (b) subjecting the molecules to digestion with a first one or more restriction enzymes, wherein said subjecting produces DNA fragments; (c) subjecting (e.g., ligating) the DNA fragments to adapters and ligase, wherein the subjecting produces a mixture of adapter-ligated DNA fragments and adapter dimers; and (d) amplifying the adapter-ligated DNA fragments to produce amplified adapter-ligated DNA fragments, wherein the method further comprises reducing the quantity of adapter dimers produced, wherein the method further comprises performing the reducing during and/or after (c) and/or (d), wherein the reducing comprises differentiating between the junction between an adapter and a DNA fragment, and the junction between an adapter and another adapter.

The first one or more restriction enzymes may comprise AclI, HindIII, MluCI, PciI, AgeI, BspMI, BfuAI, SexAI, MluI, BceAI, HpyCH4IV, HpyCH4III, BaeI, BsaXI, AflIII, SpeI, BsrI, BmrI, BglII, BspDI, PI-SceI, NsiI, AseI, CspCI, MfeI, BssS^{α}I, DraIII, EcoP15I, AlwNI, BtsIMutI, NdeI, CviAII, FatI, NlaIII, FspEI, XcmI, BstXI, PflMI, BccI, NcoI, BseYI, FauI, TspMI, XmaI, LpnPI, AclI, ClaI, SacII, HpaII, MspI, ScrFI, StyD4I, BsaJI, BslI, BtgI, NciI, AvrII, MnlI, BbvCI, SbfI, Bpu10I, Bsu36I, EcoNI, HpyAV, BstNI, PspGI, StyI, BcgI, PvuI, EagI, RsrII, BsiEI, BsiWI, BsmBI, Hpy99I, AbaSI, MspJI, SgrAI, BfaI, BspCNI, XhoI, PaeR7I, EarI, AcuI, PstI, BpmI, DdeI, SfcI, AflII, BpuEI, SmlI, Aval, BsoBI, MboII, BbsI, BsmI, EcoRI, HgaI, AatII, PflFI, Tth111I, AhdI, DrdI, SacI, BseRI, PleI, HinfI, Sau3AI, MboI, DpnII, TfiI, BsrDI, BbvI, Bts^{α}I, BstAPI, SfaNI, SphI, NmeAIII, NgoMIV, BglI, AsiSI, BtgZI, HhaI, HinPlI, BssHII, NotI, Fnu4HI, MwolI, BmtI, NheI, BspQI, BlpI, TseI, ApeKI, Bsp1286I, AlwI, BamHI, BtsCI, FokI, FseI, SfiI, NarI, PluTI, KasI, AscI, EciI, BsmFI, ApaI, PspOMI, Sau96I, KpnI, Acc65I, BsaI, HphI, BstEII, AvaII, BanI, BaeGI, BsaHI, BanII, CviQI, BciVI, SalI, BcoDI, BsmAI, ApaLI, BsgI, AccI, Tsp45I, BsiHKAI, TspRI, ApoI, NspI, BsrF^{α}I, BstYI, HaeII, EcoO109I, PpuMI, I-CeuI, I-SceI, BspHI, BspEI, MmeI, Taq^{α}I, Hpy188I, Hpy188III, XbaI, MI, PI-PspI, BsrGI, MseI, PacI, BstBI, PspXI, BsaWI, EaeI, HpyF30I, Sfr274I, or a combination thereof. In certain embodiments, it is contemplated that 1, 2, 3, 4, 5, 6, 7, 8, 9, 10 or more of these may be excluded. In some embodiments, (b) and (c) are performed in the same reaction mixture. In some embodiments, (b) is performed at a different or same temperature than (c).

In some embodiments, differentiating between the junction between an adapter and a DNA fragment, and the junction between an adapter and another adapter further comprises using an adapter designed to be digested by a second one or more restriction enzymes when in a dimerized configuration, but that is not able to be digested by the second one or more restriction enzymes when the adapter is ligated to an end of the DNA fragment. Differentiating between the junction between an adapter and a DNA fragment, and the junction between an adapter and another adapter may further comprise using an adapter designed such that, a primer for the amplifying is able to initiate polymerization at the junction between the adapter and a DNA fragment, but is not able to initiate polymerization at the function between the adapter and another adapter.

In another aspect, the present disclosure provides a method for preparing a library of nucleic acids, comprising: (a) providing a plurality of DNA molecules; (b) subjecting the molecules to digestion with a first one or more restriction enzymes, wherein said subjecting produces DNA fragments; (c) subjecting (e.g., ligating) the DNA fragments to adapters (e.g., the adapters may comprise a known sequence, a unique sequence, or a random sequence), wherein the subjecting produces a mixture of adapter-ligated DNA fragments and adapter dimers; and (d) amplifying the adapter-ligated DNA fragments to produce amplified adapter-ligated DNA fragments, subject to one or more of the following: (1) performing (d) using a primer that binds a junction between the end of the DNA fragment and the adapter, but does not bind a junction between the end of one adapter and the end of another adapter; (2) subjecting the mixture of adapter-ligated DNA fragments and adapter dimers to a second one or more restriction enzymes that digests the junction between the end of one adapter and the end of another adapter, but does not digest the junction between the end of the DNA fragment and the adapter; (3) performing (b) and (c) in the same reaction mixture, and a second one or more restriction enzymes digests the junction between the end of one adapter and the end of another adapter, but does not digest the junction between the end of the DNA fragment and the adapter; (4) the adapter is an adapter dimer by design, and a second one or more restriction enzymes digest the junction between the end of one adapter and the end of another adapter, but does not digest the junction between the end of the DNA fragment and the adapter; and/or (5) the amplifying produces amplified adapter dimers that are digested with a third one or more restriction enzymes that digests the junction between the end of one adapter and the end of another adapter.

In some embodiments, the method further comprises subjecting the adapter-ligated fragments to conditions sufficient to permit the methylated nucleic acid bases to be distinguishable from the unmethylated nucleic acid bases. The conditions sufficient to permit the methylated nucleic acid bases to be distinguishable from the unmethylated nucleic acid bases may comprise subjecting the adapter-ligated fragments to bisulfite conversion. The conditions sufficient to permit the methylated nucleic acid bases to be distinguishable from the unmethylated nucleic acid bases may comprise subjecting the adapter-ligated fragments to one or more enzymatic and/or chemical reactions, such as to oxidize the methylated cytosine nucleic acid bases and/or hydroxymethylated cytosine nucleic acid bases, followed by reduction and/or deamination of oxidation reaction products. The deamination of the oxidation reaction products may be performed with apolipoprotein B mRNA editing enzyme, catalytic polypeptide-like (APOBEC) to deaminate cytosine nucleic acid bases. The reduction and/or deamination of oxidation reaction products may be performed with pyridine borane. In some embodiments, the method further comprises performing β-glucosyltransferase treatment before the one or more enzymatic and/or chemical reactions.

In some embodiments, part or all of the amplified adapter-ligated DNA fragments are further subjected to analysis, modification, or both. The analysis may comprise sequencing, such as next generation sequencing. In some embodiments, a targeted capture is performed before the next generation sequencing to further enrich adapter-ligated fragments. In some embodiments, size selection is performed before the next generation sequencing to further enrich adapter-ligated fragments. The analysis may comprise analyzing the methylation pattern of the amplified adapter-ligated DNA fragments. The adapter may comprise a GC (in a 3′ to 5′ direction) overhang. The first one or more restriction enzymes may comprise MspI, HpaII, TaqαI or a functional analog, or a mixture thereof. The second one or more restriction enzymes may comprise one or more of BspD1, ClaI, AclI, NarI, XhoI, SmlI, HpyF30I, PaeR7I, Sfr274I, or a functional analog or a mixture thereof. In some embodiments, the ligase is T7 DNA ligase, T4 DNA ligase, T3 DNA ligase, Taq DNA ligase, or a functional analog thereof or a mixture thereof.

In some embodiments, the plurality of DNA molecules comprises cell-free DNA. In some embodiments, the method further comprises obtaining the cfDNA, such as from a sample obtained or derived from a subject or individual. The cfDNA may be enriched for molecules having one or more CpG sites. The sample may be of any kind, such as from plasma, serum, bone marrow, cerebral spinal fluid, pleural fluid, saliva, stool, or urine. In some embodiments, the method further comprises obtaining the sample from the subject or individual.

In another aspect, the present disclosure provides a method for preparing a library of nucleic acids, comprising: (a) providing a plurality of DNA molecules; (b) subjecting the molecules to digestion with a first one or more restriction enzymes, wherein the subjecting produces DNA fragments; (c) subjecting (e.g., ligating) the DNA fragments to adapters, wherein the subjecting produces a mixture of adapter-ligated DNA fragments and adapter dimers; and (d) amplifying the adapter-ligated DNA fragments to produce amplified adapter-ligated DNA fragments, wherein the amplifying uses a set of primers that binds a junction between the end of the DNA fragment and the adapter, but does not bind a junction between the end of one adapter and the end of another adapter. In some embodiments, the first one or more restriction enzymes comprise one or more of MspI, HpaII, TaqαI, or a functional analog thereof or a mixture thereof.

In some embodiments, the method further comprises performing (b) and (c) in the same reaction mixture. In some embodiments, the method further comprises subjecting the adapter-ligated fragments to conditions sufficient to permit the methylated nucleic acid bases to be distinguishable from the unmethylated nucleic acid bases. The conditions sufficient to permit the methylated nucleic acid bases to be distinguishable from the unmethylated nucleic acid bases may comprise subjecting the adapter-ligated fragments to bisulfite conversion or subjecting the adapter-ligated fragments to one or more enzymatic and/or chemical reactions (for example, to oxidize the methylated cytosine nucleic acid bases and/or hydroxymethylated cytosine nucleic acid bases, followed by reduction and/or deamination of oxidation reaction products).

In some embodiments, the oxidizing is performed with ten-eleven translocation (TET) enzymes. In some embodiments, the oxidizing is performed with potassium perruthenate. In some embodiments, the deamination of the oxidation reaction products is performed with APOBEC to deaminate cytosine nucleic acid bases, or the deamination of the oxidation reaction products may be performed with pyridine borane. In some embodiments, the method further comprises performing β-glucosyltransferase treatment before the one or more enzymatic or chemical reactions. In some embodiments, the adapter comprises a GC overhang.

In another aspect, the present disclosure provides a method for preparing a library of nucleic acids, comprising: (a) providing a plurality of DNA molecules; (b) subjecting the molecules to digestion with a first one or more restriction enzymes, wherein said subjecting produces DNA fragments; (c) subjecting (e.g., ligating) the DNA fragments to adapters (e.g., that may comprise a GC overhang), wherein the subjecting produces a mixture of adapter-ligated DNA fragments and adapter dimers; (d) subjecting the mixture of adapter-ligated DNA fragments and adapter dimers to a second one or more restriction enzymes that digests the junction between the end of one adapter and the end of another adapter, but does not digest the junction between the end of the DNA fragment and the adapter; and (e) amplifying the adapter-ligated DNA fragments to produce amplified adapter-ligated DNA fragments. In some embodiments, the method further comprises performing (b), (c), and (d) in the same reaction mixture.

In some embodiments, the first one or more restriction enzymes comprise one or more of MspI, HpaII, TaqαI, or a functional analog thereof or a mixture thereof. The second one or more restriction enzymes may comprise one or more of BspDI, ClaI, AclI, NarI, XhoI, SmlI, HpyF30I, PaeR7I, Sfr274I, or a functional analog thereof or a mixture thereof.

In some embodiments, the method further comprises subjecting the adapter-ligated fragments to conditions sufficient to permit the methylated nucleic acid bases to be distinguishable from the unmethylated nucleic acid bases, such as subjecting the adapter-ligated fragments to bisulfite conversion or subjecting the adapter-ligated fragments to one or more enzymatic and/or chemical reactions, such as to oxidize the methylated cytosine nucleic acid bases and/or hydroxymethylated cytosine nucleic acid bases, followed by reduction and/or deamination of oxidation reaction products (for example, performed with APOBEC or pyridine borane). In some embodiments, the oxidizing is performed with ten-eleven translocation (TET) enzymes. In some embodiments, the oxidizing is performed with potassium perruthenate. In some embodiments, the method further comprises performing β-glucosyltransferase treatment before the one or more enzymatic or chemical reactions. In some embodiments, the adapter comprises a GC overhang.

In another aspect, the present disclosure provides a method for preparing a library of nucleic acids, comprising: (a) providing a plurality of DNA molecules; (b) subjecting the molecules to digestion with a first one or more restriction enzyme, wherein said subjecting produces DNA fragments; (c) subjecting (e.g., ligating) the DNA fragments to adapters that are adapter dimers by design, comprising subjecting the adapter dimers to a second one or more of restriction enzymes to produce the adapters, wherein the subjecting further produces a mixture of adapter-ligated DNA fragments and adapter dimers, wherein the second one or more of restriction enzymes digest the junction between the end of one adapter and the end of another adapter, but do not digest the junction between the end of the DNA fragment and the adapter; and (d) amplifying the adapter-ligated DNA fragments to produce amplified adapter-ligated DNA fragments. In some embodiments, the method further comprises performing (b) and (c) in the same reaction mixture.

In some embodiments, the first one or more of restriction enzymes comprise one or more of MspI, HpaII, TaqαI, or a functional analog thereof or a mixture thereof. The second one or more of restriction enzymes may comprise one or more of BspDI, ClaI, AclI, NarI, XhoI, SmlI, HpyF30I, PaeR7I, Sfr274I, or a functional analog thereof or a mixture thereof. In some embodiments, the method further comprises performing (b) and (c) in the same reaction mixture.

In some embodiments, the method further comprises subjecting the adapter-ligated fragments to conditions sufficient to permit the methylated nucleic acid bases to be distinguishable from the unmethylated nucleic acid bases. The conditions may comprise subjecting the adapter-ligated fragments to bisulfite conversion, or subjecting the adapter-ligated fragments to one or more enzymatic and/or chemical reactions, such as to oxidize the methylated cytosine nucleic acid bases and/or hydroxymethylated cytosine nucleic acid bases, followed by reduction and/or deamination of oxidation reaction products (for example, with APOBEC or with pyridine borane). In some embodiments, the oxidizing is performed with ten-eleven translocation (TET) enzymes. In some embodiments, the oxidizing is performed with potassium perruthenate. In some embodiments, the method further comprises performing β-glucosyltransferase treatment before the one or more enzymatic or chemical reactions. In some embodiments, digestion by the second one or more of restriction enzymes of the adapter dimers of the second adapters produces GC overhangs.

In another aspect, the present disclosure provides a method for preparing a library of nucleic acids, comprising: (a) providing a plurality of DNA molecules; (b) subjecting the molecules to digestion with a first one or more restriction enzymes, wherein said subjecting produces DNA fragments; (c) subjecting (e.g., ligating) the DNA fragments to adapters, wherein the subjecting produces a mixture of adapter-ligated DNA fragments and adapter dimers; and (d) amplifying the adapter-ligated DNA fragments to produce amplified adapter-ligated DNA fragments and amplified adapter dimers, wherein the amplified adapter dimers are digested with a second one or more restriction enzymes that digest the junction between the end of one adapter and the end of another adapter. In some embodiments, the method further comprises performing (b) and (c) in the same reaction mixture.

In some embodiments, the first one or more of restriction enzymes comprise one or more of MspI, HpaII, TaqαI, or a functional analog thereof or a mixture thereof. The second one or more of restriction enzymes may comprise one or more of BspDI, ClaI, AclI, NarI, XhoI, SmlI, HpyF30I, PaeR7I, Sfr274I, or a functional analog thereof or a mixture thereof.

In some embodiments, the method further comprises subjecting the adapter-ligated fragments to conditions sufficient to permit the methylated nucleic acid bases to be distinguishable from the unmethylated nucleic acid bases, for example comprising subjecting the adapter-ligated fragments to bisulfite conversion, or subjecting the adapter-ligated fragments to one or more enzymatic and/or chemical reactions. The one or more enzymatic reactions may be to oxidize the methylated cytosine nucleic acid bases and/or hydroxymethylated cytosine nucleic acid bases, followed by reduction and/or deamination of oxidation reaction products, such as with APOBEC or with pyridine borane. In some embodiments, the oxidizing is performed with ten-eleven translocation (TET) enzymes. In some embodiments, the oxidizing is performed with potassium perruthenate. In some embodiments, the method further comprises performing β-glucosyltransferase treatment before the one or more enzymatic or chemical reactions. In some embodiments, the adapter comprises a GC overhang.

In another aspect, the present disclosure provides a method for preparing a library of nucleic acids, comprising: (a) providing a plurality of DNA molecules; (b) subjecting the molecules to digestion with a first restriction enzyme in the presence of a ligase, wherein said digestion produces DNA fragments; (c) subjecting (e.g., ligating) the DNA fragments to adapters, wherein the subjecting produces a mixture of adapter-ligated DNA fragments and adapter dimers, and wherein a second one or more restriction enzyme digests the junction between the end of one adapter and the end of another adapter, but does not digest the junction between the end of the DNA fragment and the adapter; and (d) amplifying the adapter-ligated DNA fragments to produce amplified adapter-ligated DNA fragments.

In some embodiments, the method further comprises performing (b) and (c) in the same reaction mixture. In some embodiments, the method further comprises subjecting the adapter-ligated fragments to bisulfite conversion. The adapter may comprise a GC overhang.

In some embodiments of a method provided herein, the restriction enzymes that digest the junction between the end of one adapter and the end of another adapter are replaced with CRISPR-associate endonuclease and a specifically designed guide RNA.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Additional objects, features, aspects and advantages of the present invention will be set forth in part in the description which follows, and in part will be obvious from the description or may be learned by practice of the invention. Various embodiments of the present disclosure will be described in sufficient detail to enable those skilled in the art to practice the invention, and it is to be understood that other embodiments may be utilized and that changes may be made without departing from the scope of the invention. The following detailed description is, therefore, not be taken in a limiting sense, and the scope of the present invention is best defined by the appended claims.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the invention are utilized, and the accompanying drawings (also “Figure” and “FIG.” herein), of which:

FIG. 1 shows an example of library production from nucleic acids, such as cell-free DNA (cfDNA) and/or genomic DNA (gDNA), that utilizes restriction enzyme digestion and adapter ligation, followed by an amplification of the produced molecules, such as with polymerase chain reaction (PCR).

FIGS. 2A-2R provide examples of adaptors that may be used in methods of the present disclosure.

FIGS. 3A-3E provide examples of adapter-adapter dimers that can be digested by a restriction enzyme.

FIG. 4 illustrates an example of a method of the present disclosure in which cfDNA is enzymatically digested, followed by adapter ligation, bisulfite conversion, and PCR amplification, wherein the PCR primers target the junctions between the adapter and the DNA fragments but not the adapter-adapter junctions.

FIG. 5 illustrates an example of a method of the present disclosure in which cfDNA is enzymatically digested, followed by adapter ligation, bisulfite conversion, and PCR amplification, wherein the adapter dimers are selectively digested by appropriate restriction enzymes.

FIG. 6 illustrates an example of a method of the present disclosure in which cfDNA is enzymatically digested in the presence of ligase and adapters, followed by bisulfite conversion, and PCR amplification, wherein one restriction enzyme digests the adapter-adapter junction and the enzyme that originally digested the starting DNA also digests ligated targeted DNA fragments lacking the adapters; the junction of adapter and targeted DNA ligation product does not have a recognition site that can be digested by either enzyme.

FIG. 7 illustrates an example of a method of the present disclosure in which cfDNA is enzymatically digested in the presence of ligase and adapters, followed by bisulfite conversion, and PCR amplification, wherein one restriction enzyme (BspDI, for example) digests the adapter-adapter junction and the enzyme (MspI, for example) that originally digested the starting DNA also digests ligated targeted DNA fragments lacking the adapters; the junction of adapter and targeted DNA ligation product does not have a recognition site that can be digested by either restriction enzymes (e.g., BspDI or MspI).

FIG. 8 illustrates an example of a method of the present disclosure in which cfDNA is enzymatically digested, followed by adapter ligation, bisulfite conversion, and PCR amplification, wherein the adapter dimers are digested after PCR amplification.

FIGS. 9A-9C illustrate results obtained from an example of a method of the present disclosure performed on 10 nanograms (ng) of cfDNA from three plasma samples. The restriction enzyme MspI was used in the reaction. Both TBE-Urea-polyacrylamide gel analysis of the library sizes (FIG. 9A) and fragment size analysis based on the sequencing data (FIG. 9B) show results of a typical reduced representation bisulfite sequencing (RRBS) library with three characteristic peaks around 68 bp, 135 bp, and 202 bp associated with Alu repeat. FIG. 9C shows a summary of the sequencing result, including: total sequencing reads; the percentage of sequencing reads that survive the trimming in the QC pipeline; percentage of duplication; R1 sequencing reads that start with CGG sequence; R2 sequencing reads that start with CGG sequence; Percentage of sequencing reads that mapped to characteristic RRBS regions.

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### I. Examples of Definitions

In keeping with long-standing patent law convention, the words “a” and “an”, when used in the present specification in concert with the word “comprising,” including the claims, denote “one or more.” Some embodiments of the present disclosure may consist of or consist essentially of one or more elements, method steps, and/or methods of the present disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “adapter dimer” as used herein refers to a molecule produced upon ligation of a first adapter molecule to a second adapter molecule.

The term “subject,” as used herein, generally refers to an individual having a biological sample that is undergoing processing or analysis. A subject can be an animal or plant. The subject can be a mammal, such as a human, dog, cat, horse, pig or rodent. The subject can be a patient, e.g., have or be suspected of having or at risk for having a disease or disorder, such as one or more cancers (e.g., brain cancer, breast cancer, cervical cancer, colorectal cancer, endometrial cancer, esophageal cancer, gastric cancer, hepatobiliary tract cancer, leukemia, liver cancer, lung cancer, lymphoma, ovarian cancer, pancreatic cancer, skin cancer, urinary tract cancer, testicular cancer, kidney cancer, sarcoma, bile duct cancer, thyroid cancer, gall bladder cancer, spleen cancer, or prostate cancer, and the cancer may or may not comprise solid tumor(s)), one or more infectious diseases, one or more genetic disorder, or one or more tumors, or any combination thereof. For subjects having or suspected of having one or more tumors, the tumors may be of one or more types. The subject may have a disease or disorder or be suspected of having the disease or disorder. The subject may have not have the disease or disorder or not be suspected of having the disease or disorder. The subject may be a healthy control. The subject may be asymptomatic for a particular disease or disorder.

The term “sample,” as used herein, generally refers to a biological sample. The samples may be taken from tissue and/or cells or from the environment of tissue and/or cells. In some examples, the sample may comprise, or be derived from, a tissue biopsy, a cell biopsy, blood (e.g., whole blood), blood plasma, serum, bone marrow, cerebral spinal fluid, pleural fluid, saliva, stool, urine, extracellular fluid, dried blood spots, cultured cells, culture media, discarded tissue, plant matter, synthetic proteins, bacterial and/or viral samples, fungal tissue, archaea, or protozoans. The sample may have been isolated from the source prior to collection. Samples may comprise forensic evidence. Non-limiting examples include a fingerprint, saliva, urine, blood, stool, semen, or other bodily fluids isolated from the primary source prior to collection. In some examples, the sample is isolated from its primary source (cells, tissue, bodily fluids such as blood, environmental samples, etc.) during sample preparation. The sample may be derived from an extinct species including but not limited to samples derived from fossils. The sample may or may not be purified or otherwise enriched from its primary source. In some embodiments, the primary source is homogenized prior to further processing. The sample may be filtered or centrifuged to remove buffy coat, lipids, or particulate matter. The sample may also be purified or enriched for nucleic acids, or may be treated with RNases or DNases. The sample may contain tissues and/or cells that are intact, fragmented, or partially degraded.

The sample may be obtained from a subject with a disease or disorder, and the subject may or may not have had a diagnosis of the disease or disorder. The subject may be in need of a second opinion. The disease or disorder may be an infectious disease, an immune disorder or disease, a cancer, a genetic disease, a degenerative disease, a lifestyle disease, or an injury. The infectious disease may be caused by bacteria, viruses, fungi, and/or parasites. Non-limiting examples of cancers include pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, thyroid cancer, gall bladder cancer, spleen cancer, and prostate cancer. Some examples of genetic diseases or disorders include, but are not limited to, cystic fibrosis, Charcot-Marie*-Tooth disease, Huntington's disease, Peutz-Jeghers syndrome, Down syndrome, Rheumatoid arthritis, and Tay-Sachs disease. Non-limiting examples of lifestyle diseases include obesity, diabetes, arteriosclerosis, heart disease, stroke, hypertension, liver cirrhosis, nephritis, cancer, chronic obstructive pulmonary disease (COPD), hearing problems, and chronic backache. Some examples of injuries include, but are not limited to, abrasion, brain injuries, bruising, burns, concussions, congestive heart failure, construction injuries, dislocation, flail chest, fracture, hemothorax, herniated disc, hip pointer, hypothermia, lacerations, pinched nerve, pneumothorax, rib fracture, sciatica, spinal cord injury, tendons ligaments fascia injury, traumatic brain injury, and whiplash. The sample may be taken before and/or after treatment of a subject with a disease or disorder. Samples may be taken before and/or after a treatment of the subject for a disease or disorder. Samples may be taken during a treatment or a treatment regimen. Multiple samples may be taken from a subject to monitor the effects of a treatment over time, including beginning from prior to the onset of the treatment. The sample may be taken from a subject known or suspected of having an infectious disease for which diagnostic antibodies may or may not be available. Samples may be taken from a subject to monitor abnormal tissue-specific cell death or organ transplantation.

The sample may be taken from a subject suspected of having a disease or a disorder. The sample may be taken from a subject experiencing unexplained symptoms, such as fatigue, nausea, weight loss, aches, pains, weakness, or memory loss. The sample may be taken from a subject having explained symptoms. The sample may be taken from a subject at risk of developing a disease or disorder because of one or more factors such as familial and/or personal history, age, environmental exposure, lifestyle risk factors, presence of other known risk factor(s), or a combination thereof.

The sample may be taken from a healthy subject or individual. In some embodiments, samples may be taken longitudinally from the same subject or individual. In some embodiments, samples acquired longitudinally may be analyzed with the goal of monitoring individual health and early detection of health issues (e.g., early diagnosis of cancer). In some embodiments, the sample may be collected at a home setting or at a point-of-care setting and subsequently transported by a mail delivery, courier delivery, or other transport method prior to analysis. For example, a home user may collect a blood spot sample through a finger prick, and the blood spot sample may be dried and subsequently transported by mail delivery prior to analysis. In some embodiments, samples acquired longitudinally may be used to monitor response to stimuli expected to impact health, athletic performance, or cognitive performance. Non-limiting examples include response to medication, dieting, and/or an exercise regimen. In some embodiments, the individual sample is multi-purpose and allows for methylation profiling to obtain clinically relevant information but also is used for information about the individual's personal or family ancestry. In some embodiments, the samples may be collected from a pregnant woman and/or her fetus.

In some embodiments, a biological sample is a nucleic acid sample including one or more nucleic acid molecules. The nucleic acid molecules may be cell-free or substantially cell-free nucleic acid molecules, such as cell-free DNA (cfDNA) or cell-free RNA (cfRNA) or a mixture thereof. The nucleic acid molecules may be derived from a variety of sources including human, mammal, non-human mammal, ape, monkey, chimpanzee, reptilian, amphibian, or avian sources. Further, samples may be extracted from variety of animal fluids containing cell-free sequences, including but not limited to blood, serum, plasma, bone marrow, vitreous, sputum, stool, urine, tears, perspiration, saliva, semen, mucosal excretions, mucus, cerebral spinal fluid, pleural fluid, amniotic fluid, and lymph fluid. The sample may be taken from an embryo, fetus, or pregnant woman. In some examples, the sample may be isolated from the mother's blood plasma. In some examples, the sample may comprise cell-free nucleic acids (e.g., cfDNA) that are fetal in origin (via a bodily sample obtained from a pregnant subject), or are derived from tissue or cells of the subject itself.

Components of the sample (including nucleic acids) may be tagged, e.g., with identifiable tags, to allow for multiplexing of samples. Some non-limiting examples of identifiable tags include: fluorophores, magnetic nanoparticles, and nucleic acid barcodes. Fluorophores may include fluorescent proteins such as GFP, YFP, RFP, eGFP, mCherry, tdtomato, FITC, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Pacific Blue, Coumarin, BODIPY FL, Pacific Green, Oregon Green, Cy3, Cy5, Pacific Orange, TRITC, Texas Red, Phycoerythrin, Allophcocyanin, or other fluorophores. One or more barcode tags may be attached (e.g., by coupling or ligating) to cell-free nucleic acids (e.g., cfDNA) in the sample prior to sequencing. The barcodes may uniquely tag the cfDNA molecules in a sample. Alternatively, the barcodes may non-uniquely tag the cfDNA molecules in a sample. The barcode(s) may non-uniquely tag the cfDNA molecules in a sample such that additional information taken from the cfDNA molecule (e.g., at least a portion of the endogenous sequence of the cfDNA molecule), taken in combination with the non-unique tag, may function as a unique identifier for (e.g., to uniquely identify against other molecules) the cfDNA molecule in a sample. For example, cfDNA sequence reads having unique identity (e.g., from a given template molecule) may be detected based at least in part on sequence information comprising one or more contiguous-base regions at one or both ends of the sequence read, the length of the sequence read, and/or the sequence of the attached barcodes at one or both ends of the sequence read. DNA molecules may be uniquely identified without tagging by partitioning a DNA (e.g., cfDNA) sample into many (e.g., at least about 50, at least about 100, at least about 500, at least about 1 thousand, at least about 5 thousand, at least about 10 thousand, at least about 50 thousand, or at least about 100 thousand) different discrete subunits (e.g., partitions, wells, or droplets) prior to amplification, such that amplified DNA molecules can be uniquely resolved and identified as originating from their respective individual input molecules of DNA.

Any number of samples may be multiplexed. For example, a multiplexed analysis may contain at least about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, or more samples, or any range derivable therein. The identifiable tags may provide a way to interrogate each sample as to its origin, or may direct different samples to segregate to different areas or a solid support.

Any number of samples may be mixed prior to analysis without tagging or multiplexing. For example, a multiplexed analysis may contain at least about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, or more samples, or any range derivable therein. Samples may be multiplexed without tagging using a combinatorial pooling design in which samples are mixed into pools in a manner that allows signal from individual samples to be resolved from the analyzed pools using computational demultiplexing.

The samples may be enriched prior to sequencing. For example, the cfDNA molecules may be selectively enriched or non-selectively enriched for one or more regions from the subject's genome or transcriptome. For example, the cfDNA molecules may be selectively enriched for one or more regions from the subject's genome or transcriptome by targeted sequence capture (e.g., using a panel), selective amplification, or targeted amplification. As another example, the cfDNA molecules may be non-selectively enriched for one or more regions from the subject's genome or transcriptome by universal amplification. In some embodiments, amplification comprises universal amplification, whole genome amplification, or non-selective amplification. The cfDNA molecules may be size selected for fragments having a length in a predetermined range. For example, size selection can be performed on DNA fragments prior to adapter ligation for lengths in a range of 40 base pairs (bp) to 250 b, or any range derivable thereinp. As another example, size selection can be performed on DNA fragments after adapter ligation for lengths in a range of 160 bp to 400 bp, or any range derivable therein.

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more, or any range derivable therein, phosphate (PO3) groups. A nucleotide can include a nucleobase, a five-carbon sugar (either ribose or deoxyribose), and one or more phosphate groups, individually or in combination.

The terms “nucleic acid molecule,” “nucleic acid sequence,” “nucleic acid fragment,” “oligonucleotide” and “polynucleotide,” as used herein, generally refer to a polynucleotide, such as deoxyribonucleotides (DNA) or ribonucleotides (RNA), or analogs and/or combinations thereof (e.g., mixture of DNA and RNA). A nucleic acid molecule may have various lengths. A nucleic acid molecule can have a length of at least 5 bases, 10 bases, 20 bases, 30 bases, 40 bases, 50 bases, 60 bases, 70 bases, 80 bases, 90, 100 bases, 110 bases, 120 bases, 130 bases, 140 bases, 150 bases, 160 bases, 170 bases, 180 bases, 190 bases, 200 bases, 300 bases, 400 bases, 500 bases, 1 kilobase (kb), 2 kb, 3, kb, 4 kb, 5 kb, 10 kb, or 50 kb, or any range derivable therein, or it may have any number of bases between any two of the aforementioned values. An oligonucleotide is typically composed of a specific sequence of four nucleotide bases: adenine (A); cytosine (C); guanine (G); and thymine (T) (uracil (U) for thymine (T) when the polynucleotide is RNA). Thus, the terms “nucleic acid molecule,” “nucleic acid sequence,” “nucleic acid fragment,” “oligonucleotide” and “polynucleotide” are at least in part intended to be the alphabetical representation of a polynucleotide molecule. Alternatively, the terms may be applied to the polynucleotide molecule itself. This alphabetical representation can be input into databases in a computer having a central processing unit and/or used for bioinformatics applications such as functional genomics and homology searching. Oligonucleotides may include one or more nonstandard nucleotide(s), nucleotide analog(s) and/or modified nucleotides.

The terms “cell-free DNA” or “cfDNA,” as used herein, generally refer to DNA that is freely circulating in fluids of a body, such as the bloodstream or plasma therefrom. In some embodiments of methods utilized herein, the cfDNA encompasses a particular type of cfDNA, such as circulating tumor DNA (ctDNA) that is tumor-derived fragmented DNA in the bloodstream that is not associated with cells. The cfDNA may be double-stranded, single-stranded, or have characteristics of both.

The term “CpG site,” as used herein, generally refers to a position along a nucleic acid molecule that includes a cytosine (C) adjacent to a guanine (G) along a 5′ to 3′ direction. The nucleic acid molecule may include at least 1, 2, 3, 4, 5, 6, 7, 8, 9, 10, 20, 30, 40, 50, 100, 500, 1000, 10000, or more, or any range derivable therein, CpG sites. Such a CpG site along the 3′ to 5′ direction of a nucleic acid molecule may be referred to as a “GpC site.”

The term “CpG island,” as used herein, generally refers to a contiguous region of genomic DNA that satisfies the criteria of (1) having a frequency of CpG dinucleotides corresponding to an “observed-to-expected ratio” greater than about 0.6; (2) having a “GC Content” greater than about 0.5; and (3) having a length of at least about 0.2 kilobases (kb), with the possible exception that repeat regions matching these criteria are excluded or masked. Criteria for identifying CpG islands are described by, for example, Gardiner-Garden et al. (J. Mol. Biol., 196:262-282, 1987), which is hereby incorporated by reference in its entirety.

The term “CpG-rich,” as used herein, generally refers to genomic regions that have high CpG content, where the majority of DNA methylation may occur. Regions of high CpG content may have a CpG content of at least 1%, 2%, 3%, 4%, 5%, 6%, 7%, 8%, 9%, 10%, or any range derivable therein, or greater. In some embodiments, such CpG content is greater than 1%. In some embodiments, CpG-rich regions may comprise CpG islands and promoter regions. CpG-rich regions may include any length (e.g., without a length restriction to be at least 0.2 kb).

The term “bisulfite conversion,” as used herein, generally refers to a biochemical process for converting unmethylated bases (e.g., cytosine bases) to uracil bases, whereby methylation information (e.g., methylated cytosine) is preserved. Examples of reagents for bisulfite conversion include sodium bisulfite, magnesium bisulfite, and trialkylammonium bisulfite.

### II. Examples of Methods

The present disclosure provides methods for preparing nucleic acid libraries having improvements for enriching informative fragments and reducing adapter dimers that can reduce the efficiency of the library preparation. In some embodiments, the source of DNA from which the libraries are generated includes DNA of any kind, particularly cell-free DNA (cfDNA). In some embodiments, the libraries are generated following DNA fragmentation generated manually. Although, in some embodiments, the starting nucleic acid material itself may comprise fragments (such as fragmentation of a natural source (from cell apoptosis or necrosis, including from cancer cell DNA)).

The present disclosure provides methods that employ a series of operations to produce the desired libraries. In some embodiments, the methods comprise operations of digesting the DNA, ligating adapters to the ends of the digested DNA, amplifying the adapter-ligated DNA, and sequencing the amplified adapter-ligated DNA, wherein at some point in the method process adapter dimers are produced as a by-product of the steps and are also reduced in number (for example, by destroying them by digestion or other means) to enhance the efficiency of the method. The methods may also comprise bisulfite conversion, for example when the methylation status of the nucleic acid is desired to be determined, including when a methylome is desired to be produced.

In some embodiments, DNA fragmentation is performed for sequencing library preparation, for example because of the limited read length of current next generation sequencing (NGS) sequencers. In previous methods for NGS library preparation, the fragmented DNA may be end-repaired and/or dA-tailed before ligating to adapters, and a typical adapter may be blunt-ended or have an overhang.

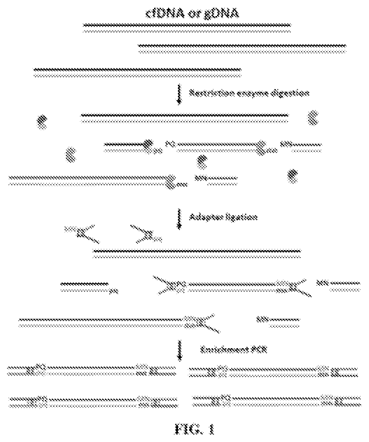

As shown in FIG. 1, previous library methods utilize an enzymatic approach, such as the use of one or more restriction enzymes, to fragment DNA with overhangs at both ends. A specially designed adapter is ligated to the fragment. Subsequently, PCR, with or without an adapter removal operation, may amplify adapter-tagged (that also may be referred to as adapter-ligated) fragments to sufficient amounts for use in NGS methods.

The digestion of DNA with restriction enzymes as an initial or early operation in the methods not only fragments DNA, but also may be utilized as a general approach to enrich DNA of interest. For example, the use of MspI enzyme in the reduced representation bisulfite sequencing (RRBS) enriches DNA fragments having CpG sites for methylation profiling. While most fragments generated from genomic DNA (gDNA) may have been cut twice by the restriction enzyme after size selection, this may not hold true for cfDNA fragments. Cell-free DNA typically comprises DNA molecules with a size distribution centered around 166 base pairs. As shown in FIG. 1, some fragments do not comprise any restriction enzyme recognition sites, and some fragments only comprise one restriction enzyme recognition site, therefore, such fragments are not informative or not as informative as compared to targeted fragments having multiple restriction enzyme recognition sites. Some restriction enzymes produce 3′ or 5′ overhangs after digestion. In FIG. 1, two representative overhangs (“PQ”, “NM”) and their complementary sequences (“pq”, “nm”) from cleavages by two restriction enzymes are shown.

In some embodiments, the present disclosure provides specially designed adapters to ligate to enzymatically digested DNA fragments for library preparation, in particular, enriching fragments with multiple restriction enzyme recognition sites from cfDNA for genomic and epi-genomic analysis. As illustrated in FIG. 1, the adapters are designed with overhangs of complementary sequences to the overhangs of restriction enzyme digested fragments (e.g., “MN”, “pq”). After adapters are ligated to targeted fragments, further library preparation processes such as PCR can only selectively amplify fragments with both ends ligated to the specially designed adapters, thus enriching the informative fragments from cfDNA for subsequent analysis, including sequencing.

One issue encountered by previous library preparation methods, such as an example shown in FIG. 1, and other library preparation methods, is the undesirable formation of numerous adapter dimers, and methods of the present disclosure overcome at least such a problem. Unlike the conventional adapters, in which their overhangs are not complementary to each other and so cannot easily form dimers, the adapters used for ligating to restriction enzyme-digested fragments can ligate to each other to form adapter dimers. Adapter dimers in the final library can be sequenced, thus, this may negatively affect the yield of sequenced reads for the targeted fragments as well as introduce spurious sequenced reads arising from adapter dimers. The adapter dimers in the produced library may be present in a large quantity and cannot be easily removed by a simple purification step, such as Ampure bead purification.

The present disclosure provides methods to avoid or reduce the quantity of adapter dimers in a library of nucleic acids, including a library produced for sequencing. In some embodiments, the methods of the present disclosure use specially designed PCR primers to selectively amplify DNA fragments with adapters on both ends and/or the use of one or more restriction enzymes to cut adapter dimers during or after adapter ligation. These methods are illustrated in the following particular applications in the use of methods of the present disclosure to produce libraries of nucleic acids. Following the preparation operations, the library may be used for any purpose, for example to profile cfDNA, including a cfDNA methylome.

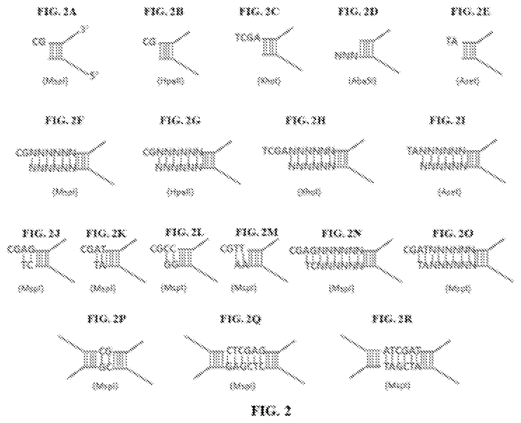

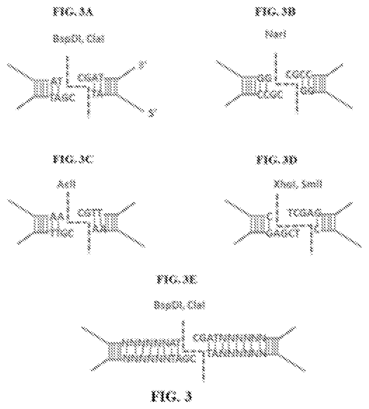

FIG. 2 illustrates examples of designed adapters that can be used to enrich informative fragments from restriction enzyme-digested cfDNA. Different restriction enzymes can be used to generate informative cfDNA fragments, including but not limited to, AcII, HindIII, MluCI, PciI, AgeI, BspMI, BfuAI, SexAI, MluI, BceAI, HpyCH4IV, HpyCH4III, BaeI, BsaXI, AflIII, SpeI, BsrI, BmrI, BglII, BspDI, PI-SceI, NsiI, AseI, CspCI, MfeI, BssSαI, DraIII, EcoP15I, AlwNI, BtsIMutI, NdeI, CviAII, FatI, NlaIII, FspEI, XcmI, BstXI, PflMI, BccI, NcoI, BseYI, FauI, TspMI, XmaI, LpnPI, AclI, ClaI, SacII, HpaII, MspI, ScrFI, StyD4I, BsaJI, BslI, BtgI, NciI, AvrII, MnlI, BbvCI, SbfI, Bpu10I, Bsu36I, EcoNI, HpyAV, BstNI, PspGI, StyI, BcgI, PvuI, EagI, RsrII, BsiEI, BsiWI, BsmBI, Hpy99I, AbaSI, MspJI, SgrAI, BfaI, BspCNI, XhoI, PaeR7I, EarI, AcuI, PstI, BpmI, DdeI, SfcI, AflII, BpuEI, SmlI, Aval, BsoBI, MboII, BbsI, BsmI, EcoRI, HgaI, AatII, PflFI, Tth111I, AhdI, DrdI, SacI, BseRI, PleI, HinfI, Sau3AI, MboI, DpnII, TfiI, BsrDI, BbvI, BtsαI, BstAPI, SfaNI, SphI, NmeAIII, NgoMIV, BglI, AsiSI, BtgZI, HhaI, HinPlI, BssHII, NotI, Fnu4HI, MwolI, BmtI, NheI, BspQI, BlpI, TseI, ApeKI, Bsp1286I, AlwI, BamHI, BtsCI, FokI, FseI, SfiI, NarI, PluTI, KasI, AscI, EciI, BsmFI, ApaI, PspOMI, Sau96I, KpnI, Acc65I, BsaI, HphI, BstEII, AvaII, BanI, BaeGI, BsaHI, BanII, CviQI, BciVI, SalI, BcoDI, BsmAI, ApaLI, BsgI, AccI, Tsp45I, BsiHKAI, TspRI, ApoI, NspI, BsrFαI, BstYI, HaeII, EcoO109I, PpuMI, I-CeuI, I-SceI, BspHI, BspEI, MmeI, TaqαI, Hpy188I, Hpy188III, XbaI, MI, PI-PspI, BsrGI, MseI, PacI, BstBI, PspXI, BsaWI, EaeI, HpyF30I, Sfr274I. In certain embodiments, it is contemplated that 1, 2, 3, 4, 5, 6, 7, 8, 9, 10 or more, or any range derivable therein, of these may be excluded.

The adapters utilized for adapter ligation in the methods of the present disclosure may be of any kind, but in some embodiments, they are specifically designed to correspond to ends of fragments to which they may be ligated. In some embodiments, the adapters are configured to be ligated to enzyme-digested nucleic acid molecules. For example, upon obtaining starting nucleic acid material (such as cfDNA from a sample), the nucleic acid is digested with one or more particular enzymes. The enzyme(s) may be selected for the purpose of enriching a particular type of nucleic acid molecule (for example, CpG-rich), for the purpose of generating fragments substantially of a certain size range, or a combination thereof. As an example, the original cfDNA may be digested with MspI. In such a case, the adapters correspond to MspI-digested DNA ends of DNA that comprise on the digested ends a CG (in a 5′ to 3′ direction) overhang. In such embodiments, the adapters have on their ends a GC (in a 3′ to 5′ direction) overhang so that they are complementary to, and are able to be ligated to, the MspI-digested DNA ends.

The adapters in FIG. 2 are merely examples of adapters that may be employed in methods of the present disclosure. Adapters for the methods of the present disclosure may comprise standard adapters with overhangs of complementary sequences to the overhangs of restriction enzyme-digested fragments (e.g., FIG. 2A, FIG. 2B, FIG. 2C, FIG. 2D, and FIG. 2E), or standard adapters with random and/or fixed sequences plus complementary sequences to the overhangs of restriction enzyme digested fragments (e.g., FIG. 2F, FIG. 2G, FIG. 2H, FIG. 2I, FIG. 2J, FIG. 2K, FIG. 2L, FIG. 2M, FIG. 2N, and FIG. 2O). In some embodiments, the adapters can be in the form of adapter-adapter dimers, including of the above adapters (e.g., FIG. 2P, FIG. 2Q, and FIG. 2R), for example; this type of adapter is specifically designed to be an adapter dimer, as opposed to those produced as a by-product of methods such as those of the present disclosure.

In some embodiments, the adapters are designed for the purpose of being able to be digested with a restriction enzyme upon generation of an adapter dimer with two molecules of adapters. In addition to this characteristic, they may be designed for the purpose of when the adapter is ligated to the end of a cfDNA fragment, the restriction enzyme that digests the adapter dimer itself is not able to digest the junction between the adapter and the end of the digested cfDNA fragment. FIGS. 3A-3E show examples of some adapter dimers that can be digested by a restriction enzyme.

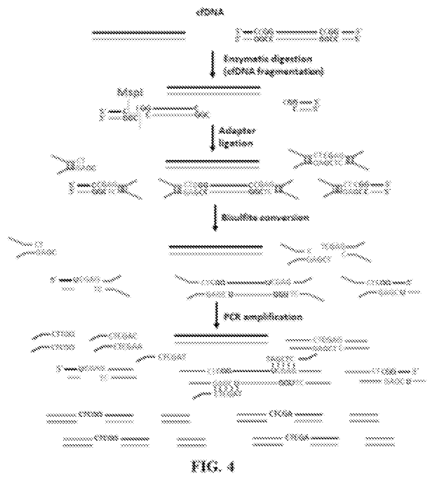

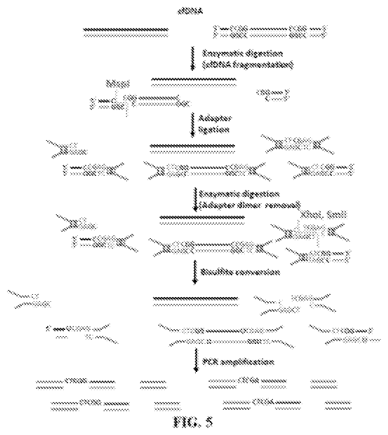

FIG. 4 illustrates the use of an example of a method for library preparation of the present disclosure, for example for methylation profiling of cfDNA for applications, such as cancer diagnosis. As an example, a restriction enzyme, MspI, is used to digest cfDNA at the recognition site CCGG. Fragments with both ends cleaved by MspI comprise CpG-rich sites that are useful for methylation profiling. The designed adapters with GC (in a 3′ to 5′ direction) overhang can ligate to the MspI cleaved ends. The digested fragments are then subjected to bisulfite treatment, such that methylated nucleic bases can be distinguished from unmethylated nucleic bases. After bisulfite conversion, PCR may be performed to enrich fragments with both ends ligated to the specially designed adapters for sequencing and methylation profiling.

In the example of FIG. 4, the adapters with GC overhangs can form adapter dimers with 5′-CTCGAG-3′ sequence at the junction of adapter dimers. However, the junction between an adapter and the ends of targeted DNA fragments as a product of restriction enzyme digestion (MspI, in this example) comprises different sequences: 5′-CTCGG-3′ (or 5′-CTTGG-3′ after bisulfite conversion if the middle C is unmethylated), etc. In some embodiments, the PCR primer is designed to recognize the junction between adapter and targeted DNA, but not the junction between adapter molecules in an adapter dimer. With this design, only ligation products with the insert of targeted DNA fragments between adapters can be amplified and enriched, for example for sequencing.

FIG. 5 illustrates an example of a method for enzymatic library preparation, for example for the purpose of cfDNA methylome profiling. In this example, after MspI digestion, targeted DNA fragments with GC overhangs at the 5′-end are directly ligated to adapters with GC overhangs at the 5′-end. Other restriction enzymes (e.g., XhoI, SmlI, and TaqαI) that recognize adapter-adapter junction sequences, but not the junction sequences of the adapter and the targeted DNA, are used to cut the adapter dimers after the ligation reaction. The restriction enzyme-digested ligation product may then be subjected to bisulfite conversion and PCR enrichment.

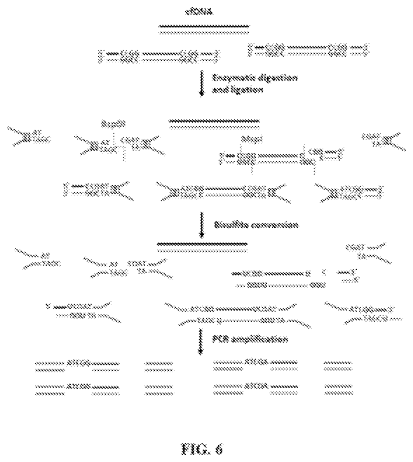

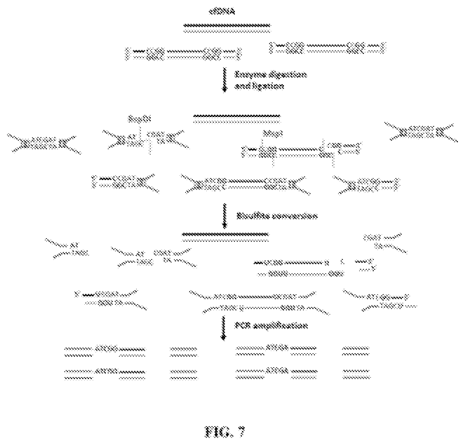

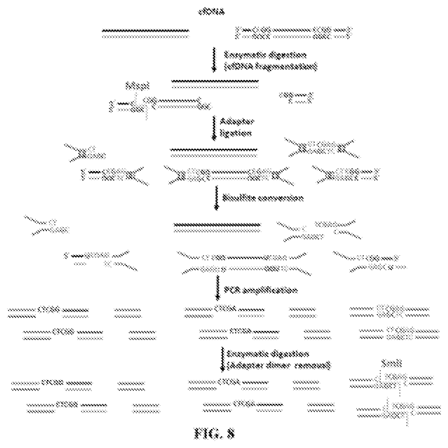

FIG. 6 illustrates an example of a method for enzymatic library preparation, including for cfDNA methylome profiling, as an example. In this example, enzymatic digestion and adapter ligation are performed in the same reaction. In this reaction, restriction enzyme MspI, as an example, generates targeted DNA fragments with GC (in a 3′ to 5′ direction) overhangs in the 5′-end. Adapters with GC overhangs at the 5′-end are ligated to the targeted DNA fragments in the presence of DNA ligase in the same reaction. Adapter and adapter, or MspI-digested fragments, can also be ligated to each other. To avoid adapter-adapter ligation to form adapter dimer production, a restriction enzyme BspDI is added into the reaction that can recognize and cut the adapter-adapter junction, whereas MspI in the mixture can digest the ligated targeted DNA fragments. However, the junction of the adapter and the targeted DNA ligation product does not have a recognition site that can be digested by a restriction enzyme, such as either BspDI or MspI. Restriction enzyme digestion and adapter ligation in the reaction can be performed at the same temperature or at different temperatures.

FIG. 7 illustrates an example of a method for enzymatic library preparation for cfDNA methylome profiling. Similar to the methods illustrated in FIG. 6, enzymatic digestion and adapter ligation are performed in the same reaction. Adapters used in this reaction are synthesized in the form of adapter-adapter dimers, for example as shown in FIG. 2P, FIG. 2Q, and FIG. 2R, for long-term stabilization. In methods of the present disclosure, the adapters that are adapter dimers used for the purpose of effecting the method steps (as opposed to being produced as part of the method steps) are specifically designed manually. The BspDI restriction enzyme can digest this type of adapter to form the product of adapters that can be used to ligate to targeted DNA fragments.

The enzymatic digestion of adapter dimers can be performed after bisulfite conversion or after PCR amplification. As shown in FIG. 8, restriction enzyme SmlI (as an example) may be utilized to cut adapter dimers after a PCR enrichment step.

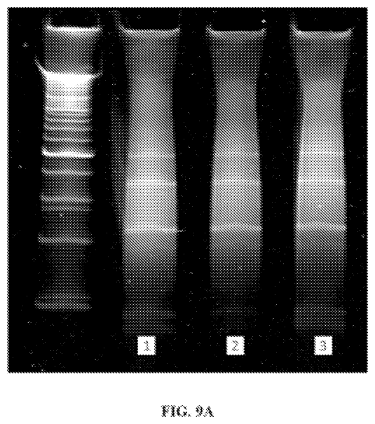

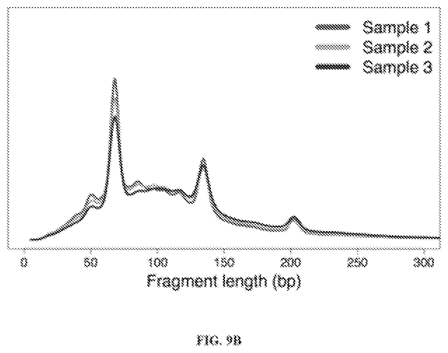

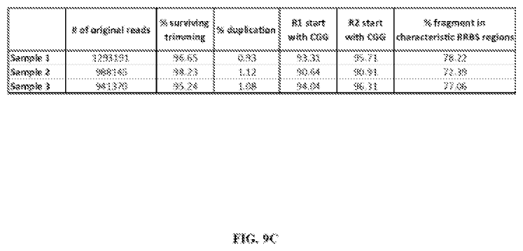

FIGS. 9A-9C illustrate an example of the preparation of a typical reduced representation bisulfite sequencing (RRBS) library from cfDNA based on a method of the present disclosure. In this example, enzymatic library preparation was performed to generate three RRBS libraries from cfDNA extracted from patient plasma. The complete protocol includes:
1. Enzymatic reaction: The enzymatic reaction solution may comprise 10 nanograms (ng) of cfDNA, H₂O, 10× CutSmart, ATP, DTT, PEG, Adapter, MspI, BspDI, and Ligase. After mixing, the solution is placed in a thermal cycler to run the following program: 17 cycles of (37° C. 30′; 25° C. 30′); 37° C. 90′; 4° C. co. The enzymatic reaction product is purified with Ampure XP bead (Beckman Coulter).
2. Bisulfite conversion: Bisulfite conversion can be performed with EpiTect Bisulfite kit (Qiagen) following manufacturer's protocol.
3. PCR amplification: The bisulfite conversion product is amplified to enrich adapter-containing fragments in the final library. The PCR reaction solution may comprise bisulfite conversion product, NEB indexing primer, NEB Universal primer, KAPA HiFI Uracil Ready Mix, and H₂O. After mixing, the solution is placed in a thermal cycler to run the following program: 98° C. 45′; 15 cycles of (98° C. 15′; 60° C. 30′; 72° C. 30′); 72° C. 60′; 4° C. co. The PCR reaction product is purified with Ampure XP bead (Beckman Coulter), and the purified library is ready for sequencing in a platform such as Illumina HiSeq 2000.

In this example, restriction enzyme MspI is used in the enzymatic reaction to digest cfDNA fragments. After bisulfite conversion and PCR amplification, this example method generates sequencing libraries that are comparable to traditional RRBS libraries prepared from intact DNA. As shown in FIGS. 9A-9C, both TBE-Urea-polyacrylamide gel analysis of the library sizes (FIG. 9A) and fragment size analysis based on the sequencing data (FIG. 9B) show results of a typical RRBS library with three characteristic peaks around 68 bp, 135 bp, and 202 bp that associated with Alu repeat. FIG. 9C shows a summary of the sequencing result, including: total sequencing reads; the percentage of sequencing reads that survive the trimming in the QC pipeline; percentage of duplication; R1 sequencing reads that start with CGG sequence; R2 sequencing reads that start with CGG sequence; Percentage of sequencing reads that mapped to characteristic RRBS regions.

Embodiments of the disclosure include at least methods for preparing a library of nucleic acids, methods for generating a plurality of polynucleotides, methods for generating double-stranded DNA, use of nucleotides to create a library, methods for preparing a sequencing library, methods of applying a sequencing library, and so forth.

Embodiments include methods involve 1, 2, 3, 4, 5, 6, 7, 8, 9, 10 or more, or any range derivable therein, of any of the following steps: providing a plurality of DNA molecules, isolating DNA molecules, connecting DNA fragments to adapters, digesting a plurality of DNA molecules, amplifying DNA molecules, ligating adapters to DNA fragments, analyzing DNA molecules of any kind, using a ligase, producing mixtures of DNA molecules, producing mixtures of ligated molecules, producing mixtures of adapter-ligated molecules or fragments, enriching a population of certain DNA molecules (including molecules that are not adapter dimers, as one example), performing certain one or more steps, distinguishing between certain DNA molecules, distinguishing between methylated and unmethylated bases, subjecting certain DNA molecules to bisulfite conversion, enzymatic reaction, and/or chemical reaction, and so forth.

### III. Nucleic Acid Molecules for Sequencing Library Preparation

In some embodiments, the nucleic acid molecules from which the sequencing library is prepared is DNA, and the DNA in some embodiments is cell-free DNA (cfDNA). The cfDNA may be obtained from a subject or individual, including a mammal. The cfDNA may be from a subject or individual in need of analysis of the cfDNA, for example to provide a determination concerning their health, such as detecting a disease condition or risk or susceptibility thereto. The cfDNA may be obtained or derived from one or more samples from the individual. The sample may be obtained or derived from plasma, serum, bone marrow, cerebral spinal fluid, pleural fluid, saliva, stool, or urine. The cfDNA from which the library is prepared may be double-stranded, single-stranded (and wherein an operation performed prior to the method may comprise polymerization of the second strand), or a mixture thereof.

In some embodiments, the nucleic acid molecules for which a library is desired to be prepared may be modified prior to utilization in methods of the present disclosure. For example, the nucleic acid molecules may be enriched for a certain type of nucleic acid molecule, a certain size of nucleic acid molecules, or a combination thereof. In some embodiments, the nucleic acid molecules are cfDNA that has been enriched, for example for a certain size of molecule and/or for molecules having one or more specific characteristics, such as those comprising one or more methylation sites.

### IV. Applications of the Sequencing Library

The present disclosure provides methods, systems, and compositions related to preparation of molecules for analysis of any kind, including for sequencing, for determining methylation quality or quantity, and so forth. In some embodiments, the molecules comprise cfDNA, and in some embodiments, the cfDNA is obtained or derived from an individual (such as blood or plasma or urine (or a combination thereof) samples from the subject or individual). In some embodiments, following library preparation, the present disclosure provides methods and systems for evaluating DNA methylation in cfDNA molecules, such as in CpG-rich regions of the cfDNA molecules.

The present disclosure is related to various aspects of methods for providing methylation information about cfDNA. Some embodiments include methods of evaluating DNA methylation in CpG-rich regions of cfDNA.

For embodiments of the present disclosure related to disease, such as cancer, detection and characterization of cfDNA in suitable samples can be an effective method for obtaining information. For example, following library preparation, the prepared sequencing library may be utilized for determining if an individual has a particular disease or medical condition or is at risk or susceptibility thereof. In an example, the individual has or is suspected of having or is at risk of having cancer, and the analysis of the library of prepared cfDNA molecules assists in determining whether the individual has or is suspected of having or is at risk of having cancer.

In some embodiments, the post-library preparation methods involve non-invasive cancer screening, including identifying the tumor tissue-of-origin. Liquid biopsy (which may also be referred to as fluid biopsy or fluid phase biopsy), e.g., blood draw, unlike traditional tissue biopsy, is useful for identifying a variety of different malignancies and may be utilized in methods of the present disclosure.

In some embodiments, at least a subset of a plurality of DNA fragments have methylated nucleic acid bases. In some embodiments, the starting cfDNA molecules may have zero, one or more CpG sites and the method comprises identifying cell-free DNA molecules as having two or three or four or more CpG sites. In some embodiments, the method further comprises, subjecting the cfDNA molecules, or derivatives thereof (including adapter ligated DNA fragments or amplified adapter ligated DNA fragments) to conditions sufficient to permit methylated nucleic acid bases in the molecules to be distinguished from unmethylated nucleic acid bases. In some embodiments, subjecting the plurality of cfDNA molecules, the plurality of DNA fragments, or derivatives thereof to sufficient conditions comprises performing bisulfite conversion on said plurality of DNA fragments. In some embodiments, subjecting the plurality of cfDNA molecules, the plurality of DNA fragments, or derivatives thereof to sufficient conditions comprises performing enzymatic and/or chemical reactions to oxidize the methylated cytosine nucleic acid bases and/or hydroxymethylated cytosine nucleic acid bases, followed by reduction and/or deamination of oxidation reaction products.

In some embodiments, the method further comprises measuring a methylation status of at least a portion of a plurality of DNA fragments or at least a portion of a plurality of adapter ligated DNA fragments, to provide a methylation profile of at least a portion of a plurality of DNA fragments. In some embodiments, the method further comprises measuring a methylation status of at least a portion of adapter ligated DNA fragments or amplified adapter ligated DNA fragments, to provide a methylation profile of the cfDNA. In some embodiments, the method further comprises processing the methylation profile against one or more references. A methylation profile may include information (including the presence and/or absence of certain methylation sites) of any number of CpG sites, CpG-rich sequences, and/or CpG islands. In some embodiments, the reference comprises a reference methylation profile of cfDNA molecules of one or more additional subjects. The subject(s) from which the reference methylation profile of cfDNA is procured may be healthy, may be cancer-free, may have cancer, or may have an elevated risk for having cancer, for example.

In some embodiments, a plurality of cfDNA molecules is obtained from a bodily sample of said subject. In some embodiments, the bodily sample is selected from the group consisting of plasma, serum, bone marrow, cerebral spinal fluid, pleural fluid, saliva, stool, sputum, nipple aspirate, biopsy, cheek scrapings, urine and a combination thereof. In some embodiments, the method further comprises processing molecules having one or more CpG sites to generate a methylation profile for a plurality of cfDNA molecules. In some embodiments, the method further comprises processing a methylation profile to generate a likelihood of the subject as having or being suspected of having a disease or disorder. In cases wherein a methylation profile from a sample from an individual is compared to one or more references, the source of the sample of the one or more references may or may not be the same source as the sample of the individual.

In some embodiments, the disease or disorder for which information is desired is selected from the group consisting of cancer, multiple sclerosis, traumatic or ischemic brain damage, diabetes, pancreatitis, Alzheimer's disease, and fetal abnormality. In some embodiments, said disease or disorder is a cancer selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, thyroid cancer, gall bladder cancer, spleen cancer, and prostate cancer.

In some embodiments, the methylation patterns of cfDNA molecules, obtained from a bodily sample of said subject, can be used to monitor abnormal tissue-specific cell death or organ transplantation.

In some embodiments, a library generated using methods or systems encompassed herein to enrich for CpG-rich regions or CpG islands in cfDNA is utilized for an application. In some embodiments, the library is assayed for one or more characteristics. The library may be assayed to determine the amount and/or location of methylation site(s) in some or all of the molecules of the library. In some embodiments, the methylation pattern is determined for at least a portion in some or all of the molecules of the library, including one or more specific sites. Methylation profiling may be performed for at least a portion of some or all of the molecules of the library.

In some embodiments, the one or more methylation sites or markers may include plasma methylation biomarkers for various specific diseases or disorders, including cancers. The differentially methylated biomarkers can be identified by comparing methylation profile data from patients with a certain disease or disorder characteristic (cancer type, stage, prognosis, treatment response, etc.) to methylation profile data from healthy controls. With a variety of methylation profiles specific to different cancers or tissue types being identified, the embodiments disclosed herein can detect many types of cancers and provide tumor location information for further specific clinical investigation based on a simple non-invasive liquid biopsy. Methylation profiles can be used to detect any disease or disorder based on a non-invasive liquid biopsy, for example.

In some embodiments, cfDNA methylation profiles can be used to diagnose a subject or a patient based at least in part on determining whether the subject has a cfDNA methylation profile indicative of a disease or disorder. In some aspects, the present disclosure provides methods of diagnosing a subject based on cfDNA methylation profile that comprise generating a cfDNA methylation profile indicative of cancer whether the patient has cancer. In some embodiments, the cfDNA methylation profile is generated by processing a biological sample from the patient that comprises cell free DNA using methods, compositions and systems encompassed herein.

In some embodiments, cfDNA methylation profile(s) can be used to diagnose a patient who has symptoms of cancer, is asymptomatic of cancer, has a family or patient history of cancer, is at risk for cancer, or who has been diagnosed with cancer. A patient may be a mammalian patient though in most embodiments the patient is a human. The cancer may be malignant, benign, metastatic, or a precancer. In still further embodiments, the cancer is melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, liver, mesothelioma, cervical, gastrointestinal, lymphoma, brain, colon, sarcoma, gall bladder thyroid, spleen, or bladder. The cancer may include a tumor comprised of tumor cells.

In some aspects, the present disclosure provides methods for treating cancer in a cancer patient following determination of a need thereof based on methods and systems herein of enriching CpG island-comprising or CpG-rich DNA for cancer diagnosis. Such methods of treating may comprise administering to the patient an effective amount of chemotherapy, radiation therapy, hormone therapy, targeted therapy, or immunotherapy (or a combination thereof) after the patient has been determined to have cancer based on methods disclosed herein. The point of origin of the cancer may be determined, in which case, the treatment is tailored to cancer of that origin. In some embodiments, tumor resection is performed as the treatment or may be part of the treatment with one of the other treatments. Examples of chemotherapeutics include, but are not limited to: alkylating agents such as bifunctional alkylators (for example, cyclophosphamide, mechlorethamine, chlorambucil, melphalan) or monofunctional alkylators (for example, dacarbazine (DTIC), nitrosoureas, temozolomide (oral dacarbazine)); anthracyclines (for example, daunorubicin, doxorubicin, epirubicin, idarubicin, mitoxantrone, and valrubicin; taxanes, which disrupt the cytoskeleton (for example, paclitaxel, docetaxel, abraxane, taxotere); epothilones; histone deacetylase inhibitors (for example, vorinostat, romidepsin); Topoisomerase I inhibitors (for example, irinotecan, topotecan); Topoisomerase II inhibitors (for example, etoposide, teniposide, tafluposide); kinase inhibitors (for example, bortezomib, erlotinib, gefitinib, imatinib, vemurafenib, and vismodegib); nucleotide analogs and nucleotide precursor analogs (for example, azacitidine. azathioprine, capecitabine, cytarabine, doxifluridine. fluorouracil, gemcitabine, hydroxyurea, mercaptopurine, methotrexate, tioguanine (formerly thioguanine); peptide antibiotics (for examples, bleomycin, actinomycin); platinum-based antineoplastics (for example, carboplatin, cisplatin, oxaliplatin); retinoids (for example, retinoin, alitretinoin, bexarotene); and, vinca alkaloids (for example, vinblastine, vincristine, vindesine, and vinorelbine). Examples of immunotherapies include, but are not limited to, cellular therapy such as dendritic cell therapy (for example, involving chimeric antigen receptor); antibody therapy (for example, Alemtuzumab, Atezolizumab, Ipilimumab, Nivolumab, Ofatumumab, Pembrolizumab, Rituximab or other antibodies with the same target as one of these antibodies, such as CTLA-4, PD-1, PD-L1, or other checkpoint inhibitors); and, cytokine therapy (for example, interferon or interleukin).

In some embodiments, methods of using cfDNA methylation profiling to diagnose a subject may further involve performing a biopsy, doing a CAT scan, doing a mammogram, performing ultrasound, or otherwise evaluating tissue suspected of being cancerous before or after determining the patient's methylation profile. In some embodiments, cancer that is found is classified in a cancer classification or staging (e.g., stage I, stage II, stage III, or stage IV).

In some embodiments, cfDNA methylation profiles obtained by methods and systems of enriching CpG islands in cfDNA is utilized for monitoring a therapy and/or monitoring tumor progression, including during and/or after treatment. For example, blood draws may be taken at various time points to monitor tumor progression throughout one or more treatment regimens, and the cfDNA therefrom may be assayed.

In some embodiments, cfDNA methylation profiles obtained by methods and systems of the present disclosure may be utilized for assessment of disease stage or as a prognostic biomarker, for example in cases where a tissue biopsy is not possible or where archived tumor samples are not available for genetic analysis.

In some embodiments, cfDNA methylation profiles obtained by methods and systems of enriching CpG-rich regions in cfDNA provided herein may be used for screening and early detection of cancer. For example, blood draws may be taken regularly from an individual without any symptoms of cancer to find cancer early or to ascertain a predisposition to cancer.

In some embodiments, cfDNA methylation profiles obtained by methods and systems of enriching CpG-rich regions in cfDNA provided herein may be used for prenatal testing of fetal DNA from maternal plasma or serum for identification of Down syndrome and other chromosomal abnormalities in a fetus.

In some embodiments, cfDNA methylation profiles obtained by methods and systems of enriching CpG-rich regions in cfDNA provided herein may be used for organ transplantation monitoring.

In some embodiments, cfDNA methylation profiles obtained by methods and systems of enriching CpG-rich regions in cfDNA provided herein may be used for diagnosis of other type of diseases such as multiple sclerosis, traumatic/ischemic brain damage, diabetes, pancreatitis, or Alzheimer's disease, or infectious diseases.

It is contemplated that any embodiment discussed in this specification can be implemented with respect to any method, system, kit, computer-readable medium, or apparatus of the invention, and vice versa. Furthermore, apparatuses used in the present disclosure can be used to achieve methods of the present disclosure.

In some embodiments, one or more CpG sites comprise two or more, three or more, or four or more CpG sites. In some embodiments, the method further comprises producing a report, such as electronically outputting a report indicative of a methylation profile. In some embodiments, the method further comprises processing a methylation profile to generate a likelihood or risk of a subject as having or being suspected of having at least one disease or disorder. In some embodiments, the disease or disorder is selected from the group consisting of cancer, multiple sclerosis, traumatic or ischemic brain damage, diabetes, pancreatitis, Alzheimer's disease, and fetal abnormality. In some embodiments, the disease or disorder is a cancer selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, thyroid cancer, spleen cancer, gall bladder cancer, and prostate cancer.

In some embodiments, one or more CpG sites comprise two or more CpG sites. In some embodiments, one or more computer processors are individually or collectively programmed to electronically output a report indicative of a methylation profile. In some embodiments, one or more computer processors are individually or collectively programmed to process a methylation profile to generate a likelihood or risk of a subject as having or being suspected of having one or more diseases or disorders. In some embodiments, said disease or disorder is selected from the group consisting of cancer, multiple sclerosis, traumatic or ischemic brain damage, diabetes, pancreatitis, Alzheimer's disease, and fetal abnormality. In some embodiments, said disease or disorder is a cancer selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, thyroid cancer, spleen cancer, gall bladder cancer, and prostate cancer.

In another aspect, the present disclosure provides a non-transitory computer-readable medium comprising machine executable code that, upon execution by one or more computer processors, implements a method for processing or analyzing a plurality of cfDNA molecules subjected to library preparation methods of the present disclosure, the method comprising: (a) retrieving a plurality of sequence reads generated by a sequencer, wherein at least a subset of said plurality of sequence reads comprises individual sequence reads comprising (i) sequences from said plurality of cfDNA molecules and (ii) adapter sequences at both ends of each of said individual sequence reads, which adapter sequences are not from said plurality of cell-free DNA molecules; (b) processing said plurality of sequence reads to (i) identify one or more sequence reads from said plurality of sequence reads having said adapter sequences at both ends, and (ii) identifying said one or more sequence reads as being associated with one or more CpG sites of said plurality of cell-free DNA molecules; and (c) using said one or more CpG sites identified in (b) to generate a methylation profile for said plurality of cell-free DNA molecules.

A library prepared from a cfDNA sample(s) from a subject may be subjected to analysis of any kind, including methylation profiling, for screening, diagnosis, prognosis, treatment selection, or treatment monitoring, for example of a tumor or of non-solid cancers. For example, analysis may suggest that patients with certain methylation profiles may respond best to surgery, chemotherapy, radiation therapy, targeted therapy, hormone therapy, immunotherapy, or a combination thereof. An accurate methylation profiling of cfDNA samples may prevent potentially ineffective treatments from being prescribed and administered to patients.

### V. Methylation Profiling of the Sequencing Library

After a library of molecules have been prepared using methods encompassed herein, methylation profiling may be performed on the enriched DNA molecules. For example, sequencing reads may be generated from the enriched DNA molecules using any suitable sequencing method. The sequencing method can be a first-generation sequencing method, such as Maxam-Gilbert or Sanger sequencing, or a high-throughput sequencing (e.g., next-generation sequencing or NGS) method. A high-throughput sequencing method may sequence simultaneously (or substantially simultaneously) at least 10,000, 100,000, 1 million, 10 million, 100 million, 1 billion, or more polynucleotide molecules. Sequencing methods may include, but are not limited to: pyrosequencing, sequencing-by-synthesis, single-molecule sequencing, nanopore sequencing, semiconductor sequencing, sequencing-by-ligation, sequencing-by-hybridization, Digital Gene Expression (Helicos), massively parallel sequencing, e.g., Helicos, Clonal Single Molecule Array (Solexa/Illumina), sequencing using PacBio, SOLiD, Ion Torrent, or Nanopore platforms, BGISEQ, or a combination thereof.

In some embodiments, the sequencing comprises whole genome sequencing (WGS). In some embodiments, the sequencing comprises whole genome bisulfite sequencing (WGBS), such as of reference DNA samples. In some embodiments, the sequencing comprises reduced representation bisulfite sequencing (RRBS), such as of reference DNA samples. In some embodiments, the sequencing comprises targeted sequencing using a panel containing a plurality of genetic loci. The sequencing may be performed at a depth sufficient to perform methylation profiling in a subject with a desired performance (e.g., accuracy, sensitivity, specificity, positive predictive value (PPV), negative predictive value (NPV), or the area under curve (AUC) of a receiver operator characteristic (ROC)). In some embodiments, the sequencing is performed at a depth of at least about 5×, at least about 10×, at least about 20×, at least about 50×, at least about 75×, at least about 100×, at least about 125×, at least about 150×, at least about 175×, or at least about 200×, or any range derivable therein.

In some embodiments, the plurality of genetic loci may correspond to coding and/or non-coding genomic regions of a genome, such as CpG islands, hypermethylated regions and/or hypomethylated regions, and/or regions proximate to such hypermethylated regions and/or hypomethylated regions. The genomic regions may correspond to cancer-associated (or tumor-associated) coding and/or non-coding genomic regions of a genome, such as cancer driver mutations or genetic variants. Genetic variants may include, for example, single nucleotide variants (SNVs), copy number variants (CNVs), insertions or deletions (indels), fusion genes, hypermethylation, and hypomethylation.

In some embodiments, performing methylation profiling of a subject may comprise aligning the cfDNA sequencing reads to a reference genome. The reference genome may comprise at least a portion of a genome (e.g., the human genome). The reference genome may comprise an entire genome (e.g., the entire human genome). In some embodiments, the reference genome may comprise a plurality of genomic regions that correspond to coding and/or non-coding genomic regions of a genome, such as CpG-rich regions, CpG islands, hypermethylated regions and/or hypomethylated regions, and/or regions proximate to such hypermethylated regions and/or hypomethylated regions. The plurality of genomic regions may correspond to cancer-associated (or tumor-associated) coding and/or non-coding genomic regions of a genome, such as cancer driver mutations or genetic variants. Genetic variants may include, for example, single nucleotide variants (SNVs), copy number variants (CNVs), insertions or deletions (indels), fusion genes, hypermethylation, and hypomethylation. The alignment may be performed using, for example, a Burrows-Wheeler algorithm or other alignment algorithm (e.g., suitable for bisulfite converted reads).

In some embodiments, performing methylation profiling in a subject may comprise generating a quantitative measure of the cfDNA sequencing reads for each of a plurality of genetic loci. Quantitative measures of the cfDNA sequencing reads may be generated, such as counts of DNA sequencing reads that are aligned with a given genetic locus (e.g., a CpG-rich region, a CpG island, a hypermethylated region, a hypomethylated region, a region proximate to a hypermethylated regions, or a region proximate to a hypomethylated region). For example, cfDNA sequencing reads having a portion or all of the sequencing read aligning with a given CpG-rich region or CpG island may be counted toward the quantitative measure for that CpG-rich region or CpG island.

A combination of patterns of specific and non-specific CpG-rich regions and/or CpG islands may form a methylation profile of a subject. Changes over time in these patterns of CpG-rich regions and/or CpG islands may be indicative of changes in methylation profile of a subject. Such changes may comprise the presence of absence of methylation of one or more particular CpG sites, an increase in the level of methylation of a specific CpG-rich site or island, a decrease in the level of methylation of a specific CpG-rich site or island, and so forth.

In some embodiments, binding measurements may be performed for methylation profiling, which may comprise assaying enriched cfDNA fragments using probes that are selective for a plurality of CpG-rich regions and/or CpG islands in the plurality of enriched cfDNA fragments. In some embodiments, the probes are nucleic acid molecules having sequence complementarity with nucleic acid sequences of CpG-rich regions and/or CpG islands. In some embodiments, the nucleic acid molecules are primers or enrichment sequences. In some embodiments, the assaying comprises use of array hybridization or polymerase chain reaction (PCR), or nucleic acid sequencing.

In some embodiments, libraries may be enriched for at least a portion of the plurality of genetic loci. In some embodiments, the enrichment may comprise amplifying a plurality of library molecules. For example, the plurality of cfDNA molecules may be amplified by selective amplification (e.g., by using a set of primers or probes comprising nucleic acid molecules having sequence complementarity with nucleic acid sequences of CpG islands). Alternatively or in combination, the plurality of cfDNA molecules may be amplified by universal amplification (e.g., by using universal primers). In some embodiments, the enrichment comprises selectively isolating at least a portion of the plurality of cfDNA molecules.

In some embodiments, performing methylation profiling in a subject comprises processing the sequence reads from the library to obtain a quantitative measure of deviation. In some embodiments, the quantitative measure of deviation is a z-score relative to one or more reference cfDNA samples. The reference cfDNA samples may be obtained from subjects having a particular methylation profile and/or from subjects not having a particular methylation profile. The reference cfDNA samples may be obtained from subjects having a cancer type or from subjects not having a cancer type (e.g., pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, thyroid cancer, gall bladder cancer, spleen cancer, and prostate cancer). The reference cfDNA samples may be obtained from subjects having a particular stage of a cancer or not having a particular stage of a cancer (including stage I, stage II, stage III, or stage IV). The reference cfDNA samples may be obtained from subjects having abnormal tissue-specific cell death.

In some embodiments, performing methylation profiling in a subject comprises determining a deviated cfDNA methylation profile of the subject when the quantitative measure of deviation satisfies a predetermined criterion. In some embodiments, the predetermined criterion is a z-score (or a quantitative measure calculated from multiple z-scores) of the methylation profile of the subject is more or less than a predetermined number. The predetermined number may be about 0.1, about 0.2, about 0.5, about 1, about 1.5, about 2, about 2.5, about 3, about 3.5, about 4, about 4.5, about 5, or more than about 5.

In some embodiments, the prepared sequencing library is analyzed for one or more particular genetic loci. In some embodiments, the plurality of genetic loci comprises CpG-rich regions, CpG islands, hypermethylated regions and/or hypomethylated regions, and/or regions proximate to such hypermethylated regions and/or hypomethylated regions. The plurality of genetic loci may comprise at least about 10 distinct genetic loci, at least about 20 distinct genetic loci, at least about 30 distinct genetic loci, at least about 40 distinct genetic loci, at least about 50 distinct genetic loci, at least about 75 distinct genetic loci, at least about 100 distinct genetic loci, at least about 500 distinct genetic loci, at least about 1 thousand distinct genetic loci, at least about 5 thousand distinct genetic loci, at least about 10 thousand distinct genetic loci, at least about 50 thousand distinct genetic loci, at least about 100 thousand distinct genetic loci, at least about 500 thousand distinct genetic loci, at least about 1 million distinct genetic loci, at least about 2 million distinct genetic loci, at least about 3 million distinct genetic loci, at least about 4 million distinct genetic loci, at least about 5 million distinct genetic loci, at least about 10 million distinct genetic loci, at least about 25 million distinct genetic loci, at least about 50 million distinct genetic loci, at least about 75 million distinct genetic loci, at least about 100 million distinct genetic loci, or more than 100 million distinct genetic loci, or any range derivable therein. The location of the distinct genetic loci may or may not be in the same gene, on the same chromosome, or on different chromosomes.

In some embodiments, determining a deviated cfDNA methylation profile of a subject is performed with a sensitivity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%, or any range derivable therein.

In some embodiments, determining a deviated cfDNA methylation profile of a subject is performed with a specificity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%, or any range derivable therein.

In some embodiments, determining a deviated cfDNA methylation profile of a subject is performed with a positive predictive value (PPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% or any range derivable therein.

In some embodiments, determining a deviated cfDNA methylation profile of a subject is performed with a negative predictive value (NPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%, or any range derivable therein.

In some embodiments, determining a deviated cfDNA methylation profile of a subject is performed with an area under curve (AUC) of a receiver operator characteristic (ROC) of at least about 0.5, at least about 0.6, at least about 0.7, at least about 0.75, at least about 0.8, at least about 0.85, at least about 0.9, at least about 0.95, at least about 0.96, at least about 0.97, at least about 0.98, or at least about 0.99, or any range derivable therein.

In some embodiments, performing methylation profiling in a subject comprises determining a normal cfDNA methylation profile of the subject when the quantitative measure of deviation satisfies a predetermined criterion. In some embodiments, the predetermined criterion is that a z-score (or a quantitative measure calculated from multiple z-scores) of the methylation profile of the subject is more or less than a predetermined number. The predetermined number may be about 0.1, about 0.2, about 0.5, about 1, about 1.5, about 2, about 2.5, about 3, about 3.5, about 4, about 4.5, about 5, or more than about 5, or any range derivable therein.

In some embodiments, determining a normal cfDNA methylation profile of the subject is performed with a sensitivity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%, or any range derivable therein.

In some embodiments, determining a normal cfDNA methylation profile of the subject is performed with a specificity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%, or any range derivable therein.

In some embodiments, determining a normal cfDNA methylation profile of the subject is performed with a positive predictive value (PPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%, or any range derivable therein.

In some embodiments, determining a normal cfDNA methylation profile of a subject is performed with a negative predictive value (NPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%, or any range derivable therein.

In some embodiments, determining a normal cfDNA methylation profile of a subject is performed with an area under curve (AUC) of a receiver operator characteristic (ROC) of at least about 0.5, at least about 0.6, at least about 0.7, at least about 0.75, at least about 0.8, at least about 0.85, at least about 0.9, at least about 0.95, at least about 0.96, at least about 0.97, at least about 0.98, or at least about 0.99, or any range derivable therein.

In some embodiments, the subject has been diagnosed with cancer or is suspected of having cancer or is at risk for having cancer. For example, the cancer may be one or more types, including: brain cancer, breast cancer, cervical cancer, colorectal cancer, endometrial cancer, esophageal cancer, gastric cancer, hepatobiliary tract cancer, leukemia, liver cancer, lung cancer, lymphoma, ovarian cancer, pancreatic cancer, skin cancer, testicular cancer, kidney cancer, sarcoma, bile duct cancer, prostate cancer, thyroid cancer, gall bladder cancer, spleen cancer, or urinary tract cancer.

In some embodiments, based on the obtained cfDNA methylation profile of the subject (e.g., determining a deviated cfDNA methylation profile or a normal cfDNA methylation profile), methods of the present disclosure include administering a therapeutically effective dose of one or more treatments to treat the disease or disorder (e.g., cancer) of the subject. In some embodiments, the treatment comprises a chemotherapy, a radiation therapy, a targeted therapy, an immunotherapy, or a combination thereof. Based on the obtained methylation profile of the subject, an existing treatment of the subject may be discontinued and another treatment may be administered to the subject. Alternatively, based on the obtained methylation profile of the subject, an existing treatment of the subject may be continued and/or another treatment may be administered to the subject. An individual may be considered refractory to one or more treatments based on outcome of the methylation profile and as a result the treatment is never given or is given but is discontinued based on the outcome of subsequent methylation profiles for the same individual or is discontinued after a certain number of doses and/or period of time has passed.

An obtained cfDNA methylation profile of a subject may be assessed to determine a diagnosis of a cancer, prognosis of a cancer, or an indication of progression or regression of a tumor in the subject. In addition, one or more clinical outcomes may be assigned based on the cfDNA methylation profile assessment or monitoring (e.g., a difference in cfDNA methylation profile between two or more time points). Such clinical outcomes may include one or more of: diagnosing the subject with a cancer comprising tumors of one or more types, diagnosing the subject with the cancer comprising tumors of one or more types and/or stages, prognosing the subject with the cancer (e.g., indicating, prescribing, or administering a clinical course of treatment (e.g., surgery, chemotherapy, radiation therapy, hormone therapy, targeted therapy immunotherapy, or other treatment) for the subject), indicating, prescribing, or administering another clinical course of action (e.g., no treatment, continued monitoring such as on a prescribed time interval basis, stopping a current treatment, switching to another treatment) for the subject, or indicating an expected survival time for the subject.

In some embodiments, determining a cfDNA methylation profile for the subject comprises determining one or more predetermined thresholds for one or more genetic loci (e.g., a plurality of CpG-rich regions and/or CpG islands). The predetermined thresholds (e.g., for each of the plurality of CpG-rich regions and/or CpG islands) may be generated by performing the cfDNA methylation profiling on one or more samples from one or more control subjects (e.g., patients known to have or not have a certain disease or disorder, patients known to have or not have a certain tumor type, patients known to have or not have a certain tumor type of a certain stage, or healthy subjects not diagnosed with or exhibiting any clinical symptoms of a disease or disorder) and identifying a suitable predetermined threshold based on the cfDNA methylation profile of the control samples.

The predetermined thresholds may be adjusted based on a desired sensitivity, specificity, positive predictive value (PPV), negative predictive value (NPV), or accuracy of determining a deviated cfDNA methylation profile or determining a normal cfDNA methylation profile of a subject. For example, the predetermined threshold may be adjusted to be lower if a high sensitivity of determining a deviated cfDNA methylation profile status of a subject is desired. Alternatively, the predetermined threshold may be adjusted to be higher if a high specificity determining a deviated cfDNA methylation profile of a subject is desired. The predetermined threshold may be adjusted so as to maximize the area under curve (AUC) of a receiver operator characteristic (ROC) of the control samples obtained from the control subjects. The predetermined threshold may be adjusted so as to achieve a desired balance between false positives (FPs) and false negatives (FNs) in determining a deviated cfDNA methylation profile of subjects.

In some embodiments, determining a cfDNA methylation profile of a subject further comprises repeating the cfDNA methylation profiling at a second later time point. The second time point may be chosen for a suitable comparison of cfDNA methylation profile relative to the first time point. Examples of second time points may correspond to a time after surgical resection, a time during treatment administration or after treatment administration to treat the disease or disorder (e.g., cancer) in the subject to monitor efficiency of the treatment, or a time after the disease or disorder (e.g., cancer) is undetectable in the subject after treatment, e.g., to monitor for residual disease or cancer recurrence in the subject.

In some embodiments, determining a cfDNA methylation profile of a subject further comprises determining a difference between a first cfDNA methylation profile and a second cfDNA methylation profile, which difference is indicative of a progression or regression of a tumor of the subject. Alternatively or in combination, the method may further comprise generating, by a computer processor, a plot of the first cfDNA methylation profile and the second cfDNA methylation profile as a function of the first time point and the second time point. The plot may be indicative of the progression or regression of the tumor of the subject. For example, the computer processor may generate a plot of the two or more cfDNA methylation profiles on a y-axis against the times corresponding to the time of collection for the data corresponding to the two or more cfDNA methylation profiles on an x-axis.

A determined difference or a plot illustrating a difference between the first cfDNA methylation profile and the second cfDNA methylation profile may be indicative of a progression or regression of a tumor of the subject. For example, if a deviation of the second cfDNA methylation profile is larger than that of the first cfDNA methylation profile, that difference may indicate, e.g., tumor progression, inefficacy of a treatment to the tumor in the subject, resistance of the tumor to an ongoing treatment, metastasis of the tumor to other sites in the subject, or residual disease or cancer recurrence in the subject. Alternatively, if a deviation of the second cfDNA methylation profile is smaller than that of the first cfDNA methylation profile, that difference may indicate, e.g., tumor regression, efficacy of a surgical resection of the tumor in the subject, efficacy of a treatment to the disease or disorder (e.g., cancer) in the subject, or lack of residual disease or cancer recurrence in the subject.

After assessing and/or monitoring cfDNA methylation profile, one or more clinical outcomes may be assigned based on the cfDNA methylation profile assessment or monitoring (e.g., a difference in cfDNA methylation profile between two or more time points). Such clinical outcomes may include one or more of: diagnosing the subject with a cancer comprising tumors of one or more types, diagnosing the subject with the cancer comprising tumors of one or more types and/or stages, prognosing the subject with the cancer (e.g., indicating, prescribing, or administering a clinical course of treatment (e.g., surgery, chemotherapy, radiation therapy, targeted therapy immunotherapy, or other treatment) for the subject, indicating, prescribing, or administering another clinical course of action (e.g., no treatment, continued monitoring such as on a prescribed time interval basis, stopping a current treatment, switching to another treatment) for the subject, or indicating an expected survival time for the subject.

### VI. Kits

Any of the compositions described herein may be comprised in a kit. In a non-limiting example, cfDNA; one or more apparatuses for collection of cfDNA; enzymes; adapters; primers; dNTPs; buffers, and other chemicals, including ATP, DTT, sodium bisulfite, and so forth may be comprised in a kit.

The components of the kits may be packaged either in aqueous media or in lyophilized form. The container means of the kits may include at least one vial, test tube, flask, bottle, or other container means, into which a component may be placed, and preferably, suitably aliquoted. Where there is more than one component in the kit, the kit also may contain a second, third or other additional container into which the additional components may be separately placed. However, various combinations of components may be comprised in a vial. The kits of the present disclosure also may include a means for containing component(s) in close confinement for commercial sale. Such containers may include blow-molded plastic containers into which the desired vials are retained.

Kits of the present disclosure may include instructions for performing methods provided herein, such as methods for digesting and enriching cfDNA and methods for subjecting the enriched cfDNA for further analysis (e.g., PCR, nucleic acid array, next-generation sequencing). Such instructions may be in physical form (e.g., printed instructions) or electronic form.

Kits of the present disclosure may include a software package or a web link to a server or cloud-computing platform for analyzing the sequencing data generated from sequencing library prepared with the kit. The analysis may provide information about the quality control of the Kits such as digestion efficiency, bisulfite conversion efficiency, and provide methylation profile of the enriched cfDNA.

Kits of the present disclosure may include a report generated by a software package provided with the kit, or by a server or cloud-computing platform. The report may provide information for (1) diagnosis and/or prophylaxis of a medical condition; (2) therapy for a medical condition; (3) therapy monitoring; and so forth. For example, the report may provide information about the presence or risk of cancer, including of a particular type of cancer.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method for preparing a library of nucleic acids, comprising:
(a) digesting a plurality of DNA molecules with a first one or more restriction enzymes to produce DNA fragments;
(b) ligating adapters to the DNA fragments by incubating with ligase to produce a mixture of adapter-ligated DNA fragments and adapter dimers;
(c) amplifying the adapter-ligated DNA fragments to produce amplified adapter-ligated DNA fragments; and
(d) reducing the quantity of the adapter dimers either after or during (b) and/or after (c), wherein the reducing comprises differentiating between the junction between an adapter and a DNA fragment, and the junction between an adapter and another adapter.

2. The method of claim 1, wherein the first one or more restriction enzymes comprise AcII, HindIII, MluCI, PciI, AgeI, BspMI, BfuAI, SexAI, MluI, BceAI, HpyCH4IV, HpyCH4III, BaeI, BsaXI, AflIII, SpeI, BsrI, BmrI, BglII, BspDI, PI-SceI, NsiI, AseI, CspCI, MfeI, BssS^{α}I, DraIII, EcoP15I, AlwNI, BtsIMutI, NdeI, CviAII, FatI, NlaIII, FspEI, XcmI, BstXI, PflMI, BccI, NcoI, BseYI, FauI, TspMI, XmaI, LpnPI, AclI, ClaI, SacII, HpaII, MspI, ScrFI, StyD4I, BsaJI, BslI, BtgI, NciI, AvrII, MnlI, BbvCI, SbfI, Bpu10I, Bsu36I, EcoNI, HpyAV, BstNI, PspGI, StyI, BcgI, PvuI, EagI, RsrII, BsiEI, BsiWI, BsmBI, Hpy99I, AbaSI, MspJI, SgrAI, BfaI, BspCNI, XhoI, PaeR7I, EarI, AcuI, PstI, BpmI, DdeI, SfcI, AflII, BpuEI, SmlI, Aval, BsoBI, MboII, BbsI, BsmI, EcoRI, HgaI, AatII, PflFI, Tth111I, AhdI, DrdI, SacI, BseRI, PleI, HinfI, Sau3AI, MboI, DpnII, TfiI, BsrDI, BbvI, Bts^{α}I, BstAPI, SfaNI, SphI, NmeAIII, NgoMIV, BglI, AsiSI, BtgZI, HhaI, HinPlI, BssHII, NotI, Fnu4HI, MwoI, BmtI, NheI, BspQI, BlpI, TseI, ApeKI, Bsp1286I, AlwI, BamHI, BtsCI, FokI, FseI, SfiI, Nan, PluTI, KasI, AscI, EciI, BsmFI, ApaI, PspOMI, Sau96I, KpnI, Acc65I, BsaI, HphI, BstEII, AvaII, BanI, BaeGI, BsaHI, BanII, CviQI, BciVI, SalI, BcoDI, BsmAI, ApaLI, BsgI, AccI, Tsp45I, BsiHKAI, TspRI, ApoI, NspI, BsrF^{α}I, BstYI, HaeII, EcoO109I, PpuMI, I-CeuI, I-SceI, BspHI, BspEI, MmeI, Taq^{α}I, Hpy188I, Hpy188III, XbaI, BclI, PI-PspI, BsrGI, MseI, PacI, BstBI, PspXI, BsaWI, EaeI, HpyF30I, Sfr274I, or a combination thereof.

3. The method of claim 1 or 2, further comprising performing (a) and (b) in the same reaction mixture.

4. The method of claim 3, wherein (a) is performed at a different temperature than (b).

5. The method of claim 3, wherein (a) is performed at the same temperature as (b).

6. The method of any one of claims 1-5, wherein differentiating between the junction between an adapter and a DNA fragment, and the junction between an adapter and another adapter further comprises using an adapter designed to be digested by a second one or more restriction enzymes when in a dimerized configuration, but that is not able to be digested by the second one or more restriction enzymes when the adapter is ligated to an end of the DNA fragment.

7. The method of any one of claims 1-6, wherein (d) comprises utilizing primers during the amplifying that are capable of initiating polymerization at the junction between the adapter and a DNA fragment, but not able to initiate polymerization at the junction between the adapter and another adapter.

8. A method for preparing a library of nucleic acids, comprising:
(a) digesting a plurality of DNA molecules with a first one or more restriction enzymes to produce DNA fragments;
(b) ligating adapters to the DNA fragments by incubating with ligase to produce a mixture of adapter-ligated DNA fragments and adapter dimers; and
(c) amplifying the adapter-ligated DNA fragments to produce amplified adapter-ligated DNA fragments,
subject to one or more of the following:
(1) performing (c) using a primer or primers that bind a junction between the end of the DNA fragment and the adapter, but does not bind a junction between the end of one adapter and the end of another adapter;
(2) digesting the mixture of adapter-ligated DNA fragments and adapter dimers with a second one or more restriction enzymes that digest the junction between the end of one adapter and the end of another adapter, but do not digest the junction between the end of the DNA fragment and the adapter;
(3) performing (a) and (b) in the same reaction mixture, and further comprising digesting the mixture with a second one or more restriction enzymes that digest the junction between the end of one adapter and the end of another adapter, but do not digest the junction between the end of the DNA fragment and the adapter;
(4) the adapter is an adapter dimer by design, and further comprising digesting the mixture of adapter-ligated DNA fragments and adapter dimers with a second one or more restriction enzymes that digest the junction between the end of one adapter and the end of another adapter, but do not digest the junction between the end of the DNA fragment and the adapter; and/or
(5) (c) produces amplified adapter dimers that are digested with a third one or more restriction enzymes that digest the junction between the end of one adapter and the end of another adapter.

9. The method of claim 8, further comprising distinguishing between methylated nucleic acid bases and unmethylated nucleic acid bases in the adapter-ligated fragments.

10. The method of claim 9, further comprising subjecting the adapter-ligated fragments to bisulfite conversion.

11. The method of claim 9 or 10, further comprising subjecting the adapter-ligated fragments to one or more enzymatic and/or chemical reactions.

12. The method of claim 11, further comprising oxidizing the methylated cytosine nucleic acid bases and/or hydroxymethylated cytosine nucleic acid bases to produce oxidation reaction products, followed by reducing and/or deaminating the oxidation reaction products.

13. The method of claim 12, wherein the oxidizing is performed with a ten-eleven translocation (TET) enzyme.

14. The method of claim 12, wherein the oxidizing is performed with potassium perruthenate.

15. The method of claim 12, wherein the deaminating of oxidation reaction products is performed with apolipoprotein B mRNA editing enzyme, catalytic polypeptide-like (APOBEC).

16. The method of claim 12, wherein the reducing and/or deaminating of oxidation reaction products is performed with pyridine borane.

17. The method of any one of claims 11-16, further comprising performing β-glucosyltransferase treatment before the one or more enzymatic and/or chemical reactions.

18. The method of any one of claims 8-17, wherein part or all of the amplified adapter-ligated DNA fragments are analyzed, modified, or both.

19. The method of claim 18, wherein the analysis comprises sequencing.

20. The method of claim 19, wherein the sequencing is next generation sequencing.

21. The method of claim 20, further comprising performing targeted capture before the next generation sequencing to further enrich adapter-ligated fragments.

22. The method of claim 20 or 21, further comprising performing size selection before the next generation sequencing to further enrich adapter-ligated fragments.

23. The method of any one of claims 18-22, further comprising analyzing the amplified adapter-ligated DNA fragments to produce a methylation profile.

24. The method of any one of claims 8-23, wherein in (1), (2), (3), or (5), the adapter comprises a GC (in a 3′ to 5′ direction) overhang.

25. The method of any one of claims 8-24, wherein the first one or more restriction enzymes comprise MspI, HpaII, TaqαI, or a functional analog thereof or a mixture thereof.

26. The method of any one of claims 8-25, wherein the second one or more restriction enzymes comprise one or more of BspD1, XhoI, SmlI, HpyF30I, PaeR7I, Sfr274I, or a functional analog thereof or a mixture thereof.

27. The method of any one of claims 8-26, wherein the ligase is T7 DNA ligase, T4 DNA ligase, T3 DNA ligase, Taq DNA ligase, or a functional analog thereof or a mixture thereof.

28. The method of any one of claims 8-27, wherein the plurality of DNA molecules comprises cell-free DNA.

29. The method of claim 28, further comprising obtaining the cfDNA.

30. The method of claim 29, wherein the cfDNA is obtained or derived from a sample from a subject or individual.

31. The method of claim 30, wherein the sample is obtained or derived from plasma, serum, bone marrow, cerebral spinal fluid, pleural fluid, saliva, stool, or urine.

32. The method of claim 30 or 31, further comprising obtaining the sample from the subject or individual.

33. The method of any one of claims 8-32, wherein the adapter comprises a known sequence.

34. The method of any one of claims 8-32, wherein the adapter comprises a unique sequence.

35. The method of any one of claims 8-34, wherein the nucleic acids are enriched for molecules having one or more CpG sites.

36. A method for preparing a library of nucleic acids, comprising:
(a) digesting a plurality of DNA molecules with a first one or more restriction enzymes to produce DNA fragments;
(b) ligating adapters to the DNA fragments by incubating with ligase to produce a mixture of adapter-ligated DNA fragments and adapter dimers; and
(c) amplifying the adapter-ligated DNA fragments to produce amplified adapter-ligated DNA fragments by utilizing one or more primers that bind a junction between the end of the DNA fragment and the adapter, but do not bind a junction between the end of one adapter and the end of another adapter.

37. The method of claim 36, wherein the first one or more restriction enzymes comprise one or more of MspI, HpaII, TaqαI, or a functional analog thereof or a mixture thereof.

38. The method of claim 36 or 37, further comprising performing (a) and (b) in the same reaction mixture.

39. The method of any one of claims 36-38, further comprising distinguishing between methylated nucleic acid bases and unmethylated nucleic acid bases in the adapter-ligated fragments.

40. The method of claim 39, further comprising subjecting the adapter-ligated fragments to bisulfite conversion.

41. The method of claim 39 or 40, further comprising subjecting the adapter-ligated fragments to one or more enzymatic and/or chemical reactions.

42. The method of claim 41, further comprising oxidizing the methylated cytosine nucleic acid bases and/or hydroxymethylated cytosine nucleic acid bases to produce oxidation reaction products, followed by reducing and/or deaminating the oxidation reaction products.

43. The method of claim 42, wherein the oxidizing is performed with ten-eleven translocation (TET) enzymes.

44. The method of claim 42, wherein the oxidizing is performed with potassium perruthenate.

45. The method of claim 42, wherein the reducing and/or deaminating of oxidation reaction products is performed with APOBEC.

46. The method of claim 42, wherein the reducing and/or deaminating of oxidation reaction products is performed with pyridine borane.

47. The method of any one of claims 41-46, further comprising performing β-glucosyltransferase treatment before the one or more enzymatic or chemical reactions.

48. The method of any one of claims 36-47, wherein the adapter comprises a GC overhang.

49. A method for preparing a library of nucleic acids, comprising:
(a) digesting a plurality of DNA molecules with a first one or more restriction enzymes to produce DNA fragments;
(b) ligating adapters to the DNA fragments by incubating with ligase to produce a mixture of adapter-ligated DNA fragments and adapter dimers;
(c) digesting the mixture of adapter-ligated DNA fragments and adapter dimers with a second one or more restriction enzymes that digest the junction between the end of one adapter and the end of another adapter, but do not digest the junction between the end of the DNA fragment and the adapter; and
(d) amplifying the adapter-ligated DNA fragments to produce amplified adapter-ligated DNA fragments.

50. The method of claim 49, wherein the first one or more restriction enzymes comprise one or more of MspI, HpaII, TaqαI, or a functional analog thereof or a mixture thereof.

51. The method of claim 49 or 50, wherein the second one or more restriction enzymes is one or more of BspDI, ClaI, AclI, NarI, XhoI, SmlI, HpyF30I, PaeR7I, Sfr274I, or a functional analog thereof or a mixture thereof.

52. The method of any one of claims 49-51, further comprising performing (a), (b), and (c) in the same reaction mixture.

53. The method of any one of claims 49-52, further comprising distinguishing between the methylated nucleic acid bases and the unmethylated nucleic acid bases in the adapter-ligated fragments.

54. The method of claim 53, further comprising subjecting the adapter-ligated fragments to bisulfite conversion.

55. The method of claim 53, further comprising subjecting the adapter-ligated fragments to one or more enzymatic and/or chemical reactions.

56. The method of claim 55, further comprising oxidizing the methylated cytosine nucleic acid bases and/or hydroxymethylated cytosine nucleic acid bases to produce oxidation reaction products, followed by reducing and/or deaminating the oxidation reaction products.

57. The method of claim 56, wherein the oxidizing is performed with ten-eleven translocation (TET) enzymes.

58. The method of claim 56, wherein the oxidizing is performed with potassium perruthenate.

59. The method of claim 56, wherein the reducing and/or deaminating of the oxidation reaction products is performed with APOBEC.

60. The method of claim 56, wherein the reducing and/or deaminating of the oxidation reaction products is performed with pyridine borane.

61. The method of any one of claims 55-60, further comprising performing β-glucosyltransferase treatment before the one or more enzymatic and/or chemical reactions.

62. The method of any one of claims 49-61, wherein the adapter comprises a GC overhang.

63. A method for preparing a library of nucleic acids, comprising:
(a) digesting a plurality of DNA molecules with a first one or more restriction enzymes to produce DNA fragments;
(b) ligating by incubating with ligase DNA fragments and first adapters that are adapter dimers by design and subjecting the adapter dimers by design to a second one or more of restriction enzymes to produce second adapters and also to produce a mixture of DNA fragments ligated to the second adapters and adapter dimers of the second adapters, wherein the second one or more of restriction enzymes digest the junction between the end of one second adapter and the end of another second adapter, but do not digest the junction between the end of the DNA fragment and the second adapter; and
(c) amplifying the DNA fragments ligated to the second adapters to produce amplified adapter-ligated DNA fragments.

64. The method of claim 63, wherein the first one or more restriction enzymes comprise one or more of MspI, HpaII, TaqαI, or a functional analog thereof or a mixture thereof.

65. The method of claim 63 or 64, wherein the second one or more restriction enzymes comprise one or more of BspDI, ClaI, AclI, NarI, XhoI, SmlI, HpyF30I, PaeR7I, Sfr274I, or a functional analog thereof or a mixture thereof.

66. The method of any one of claims 63-65, further comprising performing (a) and (b) in the same reaction mixture.

67. The method of any one of claims 63-66, further comprising distinguishing between methylated nucleic acid bases and unmethylated nucleic acid bases in the DNA fragments ligated to the second adapters.

68. The method of claim 67, further comprising subjecting the DNA fragments ligated to the second adapters to bisulfite conversion.

69. The method of claim 67, further comprising subjecting the DNA fragments ligated to the second adapters to one or more enzymatic and/or chemical reactions.

70. The method of claim 69, further comprising oxidizing the methylated cytosine nucleic acid bases and/or hydroxymethylated cytosine nucleic acid bases to produce oxidation reaction products, followed by reducing and/or deaminating the oxidation reaction products.

71. The method of claim 70, wherein the oxidizing is performed with ten-eleven translocation (TET) enzymes.

72. The method of claim 70, wherein the oxidizing is performed with potassium perruthenate.

73. The method of claim 70, wherein the reducing and/or deaminating of the oxidation reaction products is performed with APOBEC.

74. The method of claim 70, wherein the reducing and/or deaminating of the oxidation reaction products is performed with pyridine borane.

75. The method of any one of claims 69-74, further comprising performing β-glucosyltransferase treatment before the one or more enzymatic or chemical reactions.

76. The method of any one of claims 63-75, wherein digestion by the second one or more of restriction enzymes of the adapter dimers of the second adapters produces GC overhangs.

77. A method for preparing a library of nucleic acids, comprising:
(a) digesting a plurality of DNA molecules with a first one or more restriction enzymes to produce DNA fragments;
(b) ligating adapters to the DNA fragments to produce a mixture of adapter-ligated DNA fragments and adapter dimers;
(c) amplifying the adapter-ligated DNA fragments to produce a mixture of amplified adapter-ligated DNA fragments and amplified adapter dimers; and
(d) digesting the mixture of amplified adapter-ligated DNA fragments and amplified adapter dimers with a second one or more restriction enzymes that digest the junction between the end of one adapter and the end of another adapter, but do not digest the junction between the end of the DNA fragment and the adapter.

78. The method of claim 77, wherein the first one or more of restriction enzymes comprise one or more of MspI, HpaII, TaqαI, or a functional analog thereof or a mixture thereof.

79. The method of claim 77 or 78, wherein the second one or more of restriction enzymes comprises one or more of BspDI, ClaI, AclI, NarI, XhoI, SmlI, HpyF30I, PaeR7I, Sfr274I, or a functional analog thereof or a mixture thereof.

80. The method of any one of claims 77-79, further comprising performing (a) and (b) in the same reaction mixture.

81. The method of any one of claims 77-80, further comprising distinguishing between the methylated nucleic acid bases and unmethylated nucleic acid bases in the adapter-ligated DNA fragments.

82. The method of claim 81, further comprising subjecting the adapter-ligated fragments to bisulfite conversion.

83. The method of claim 81, further comprising subjecting the adapter-ligated fragments to one or more enzymatic and/or chemical reactions.

84. The method of claim 83, further comprising oxidizing the methylated cytosine nucleic acid bases and/or hydroxymethylated cytosine nucleic acid bases to produce oxidation reaction products, followed by reducing and/or deaminating of the oxidation reaction products.

85. The method of claim 84, wherein the oxidizing is performed with ten-eleven translocation (TET) enzymes.

86. The method of claim 84, wherein the oxidizing is performed with potassium perruthenate.

87. The method of claim 84, wherein the reducing and/or deaminating of the oxidation reaction products is performed with APOBEC.

88. The method of claim 84, wherein the reducing and/or deaminating of the oxidation reaction products is performed with pyridine borane.

89. The method of any one of claims 83-88, further comprising performing β-glucosyltransferase treatment before the one or more enzymatic and/or chemical reactions.

90. The method of any one of claims 77-89, wherein the adapter comprises a GC overhang.
